(19) United States
(12) Patent Application Publication
(10) Pub. No.: US 2016/0015817 A1
(43) Pub. Date: Jan. 21, 2016

(54) METHODS OF TREATMENT OF PEDIATRIC SOLID TUMOR

(71) Applicant: ABRAXIS BIOSCIENCE, LLC, Los Angeles, CA (US)

(72) Inventors: Bouchra BENETTAIB, Yverdon-les-Bains (CH); Ileana ELIAS, Toronto (CA); Markus RENSCHLER, Redwood City, CA (US); Neil P. DESAI, Los Angeles, CA (US)

(73) Assignee: ABRAXIS BIOSCIENCE, LLC, Los Angeles, CA (US)

(21) Appl. No.: 14/771,783

(22) PCT Filed: Mar. 10, 2014
(86) PCT No.: PCT/US2014/022541

§ 371 Date: Aug. 31, 2015

Related U.S. Application Data
(60) Provisional application No. 61/780,658, filed on Mar. 13, 2013.
(60) Provisional application No. 61/805,817, filed on Mar. 27, 2013.
(60) Provisional application No. 61/829,940, filed on May 31, 2013.
(60) Provisional application No. 61/909,868, filed on Nov. 27, 2013.

(51) Int. Cl. A61K 47/42; A61K 9/50; A61K 31/337

(52) U.S. Cl. CPC A61K 47/42; A61K 31/337; A61K 9/5052

(57) ABSTRACT

The present invention provides methods and compositions for treating pediatric solid tumor by administering a composition comprising nanoparticles that comprise a taxane and an albumin.

### CROSS REFERENCE TO RELATED APPLICATIONS

This application claims priority benefit to U.S. Provisional Application No. 61/780,658, entitled “Methods of Treatment of Pediatric Solid Tumor,” filed Mar. 13, 2013; U.S. Provisional Application No. 61/805,817, entitled “Methods of Treatment of Pediatric Solid Tumor,” filed Mar. 27, 2013; U.S. Provisional Application No. 61/829,940, entitled “Methods of Treatment of Pediatric Solid Tumor,” filed May 31, 2013; and to U.S. Provisional Application No. 61/909,868, entitled “Methods of Treatment of Pediatric Solid Tumor,” filed Nov. 27, 2013, the contents of which are incorporated by reference herein in their entirety.

### TECHNICAL FIELD

The present invention relates to methods and compositions for the treatment of pediatric solid tumors by administering compositions comprising nanoparticles that comprise a taxane and an albumin.

### BACKGROUND

Childhood cancers differ from adult cancers in regards to incidence, origins, etiology, response to treatment, and outcomes. About 538 out of 100,000 adults are diagnosed with cancer annually. Epithelial cancers (carcinomas) are most common in adults and may result from diet, lifestyle, and environmental carcinogens. Pediatric cancers are diagnosed in about 16 out of 100,000 children and teens below the age of 15 every year. Pediatric cancers are commonly of embryonal origin (i.e. characterized by the proliferation of tissue that is normally seen in only in the developing embryo) or derived from primitive mesenchymal tissue (e.g., sarcomas). Very little is known about the causes of most pediatric cancers. Solid tumors make up about 30% of all pediatric cancers. The most common types of solid tumors in children include brain tumors, neuroblastoma, rhabdomyosarcoma, and osteosarcoma. Such solid tumors rarely occur in adults.

Albumin-based nanoparticle compositions have been developed as a drug delivery system for delivering substantially water insoluble drugs such as a taxanes. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as pancreatic cancer and melanoma.

The disclosures of all publications, patents, patent applications and published patent applications referred to herein are hereby incorporated herein by reference in their entirety.

### BRIEF SUMMARY OF THE INVENTION

In one aspect, the invention provides a method of treating a solid tumor in a human individual, comprising administering to the individual an effective amount of a composition comprising nanoparticles comprising a taxane and albumin, wherein the individual is no more than about 21 years old (such as no more than about 18 years old). In some embodiments, the solid tumor is an abdominal tumor, a soft tissue tumor, a bone tumor, or an eye tumor. In some embodiments, the solid tumor is a soft tissue sarcoma. In some embodiments, the solid tumor is rhabdomyosarcoma. In some embodiments, the solid tumor is neuroblastoma.

In some embodiments according to (or as applied to) any of the embodiments above, the individual has had a prior treatment. In some alternative embodiments, the individual is resistant or refractory to the prior treatment. In some alternative embodiments, the individual has progressed on the prior treatment. In some alternative embodiments, the individual has a recurrent solid tumor. In some alternative embodiments, the prior treatment is a taxane-based therapy.

In some embodiments according to (or as applied to) any of the embodiments above, the composition comprising nanoparticles comprising taxane and albumin is administered parenterally (such as intravenously).

In some embodiments according to (or as applied to) any of the embodiments above, the taxane is paclitaxel.

In some embodiments according to (or as applied to) any of the embodiments above, the nanoparticles in the composition have an average diameter of no greater than about 200 nm.

In some embodiments according to (or as applied to) any of the embodiments above, the taxane in the nanoparticles is coated with albumin.

In some embodiments according to (or as applied to) any of the embodiments above, the weight ratio of the albumin to the taxane in the composition is about 9:1 or less, such as about 9:1.

In some embodiments according to (or as applied to) any of the embodiments above, the nanoparticle composition is administered at about 60 mg/m² to about 300 mg/m², such as about 90 mg/m² to about 150 mg/m², for example about 100 mg/m².

In some embodiments according to any of the embodiments above, the individual is no more than about 18 years old, such as about 6 months to about 5 years old, about 5 years old to about 9 years old, about 10 to about 15 years old.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1A shows the effect of Abraxane® on cell viability of three rhabdomyosarcoma cell lines, RH4, RH30, and RD.

FIG. 1B shows the effect of Abraxane® on cell viability of the osteosarcoma cell line, KHOS.

FIG. 1C shows the effect of Abraxane® on cell viability of seven neuroblastoma cell lines, CHLA-20, CHLA-15, CHLA-90, LAN-5, SK-N-BE(2), BE(2)C, and SH-SY5Y.

FIG. 2A shows effect of Abraxane® treatment on cell viability compared to paclitaxel treatment of the neuroblastoma cell lines, SK-N-BE(2) and SY5Y.

FIG. 2B shows effect of Abraxane® treatment on cell viability compared to paclitaxel treatment of the neuroblastoma cell lines, CHLA-20 and LAN-5.

FIG. 2C shows the effect of Abraxane® treatment on cell viability compared to paclitaxel treatment of the neuroblastoma cell lines, CHLA-15 and CHLA-90.

FIG. 3 shows annexin V-FITC fluorescence staining to determine the effect of Abraxane® treatment on apoptosis of RH4 cells.

FIG. 4A shows the results of experiments conducted to determine plasma and intratumor, paclitaxel or Abraxane®, concentrations in RH4 cells following treatment.

FIG. 4B shows the results of experiments conducted to determine plasma and intratumor, paclitaxel or Abraxane®, concentrations in SK-N-BE(2) cells following treatment.

FIG. 5A shows the effect of Abraxane® or paclitaxel on tumor volume in the RH4 xenograft model.

FIG. 5B shows the effect of Abraxane® or paclitaxel on mouse body weight in the RH4 xenograft model.

FIG. 5C shows the effect of paclitaxel and Abraxane® on tumor volume in the RD xenograft model.

FIG. 6A shows the effect of paclitaxel and Abraxane® on tumor volume in the relapsed RH4 xenograft model.

FIG. 6B shows the effect of Abraxane® on tumor volume in the relapsed RH4 xenograft model.

FIG. 7A shows the effect of Abraxane® on tumor volume in the SK-N-BE(2) xenograft model.

FIG. 7B shows the effect of Abraxane® on tumor volume in the CHLA-20 xenograft model.

FIG. 8A shows the effect of Abraxane® on animal survival in the SK-N-BE(2) metastatic model.

FIG. 8B shows the effect of Abraxane® on animal body weight in the SK-N-BE(2) metastatic model.

FIG. 9 shows the cleaved caspase-3 staining of SK-N-BE(2) tumor cells treated with saline (control), Abraxane®, or Taxol® (paclitaxel).

FIG. 10 shows the phospho-histone H3 staining of SK-N-BE(2) tumor cells treated with saline (control), Abraxane®, or Taxol® (paclitaxel).

FIG. 11 shows the phospho-histone H3 staining of RH4 tumor cells treated with saline (control) or Abraxane®.

FIG. 12 shows SPARC and PTEN expression in a panel of 8 neuroblastoma cell lines by Western blot.

FIG. 13A shows primary Ewing's sarcoma including gross appearance.

FIG. 13B shoes hematoxylin and eosin staining of Ewings sarcoma cells at 400× magnification.

FIG. 13C shows diffuse expression of SPARC in Ewings sarcoma at 100× magnification.

FIGS. 14A and 14B show tumor growth and survival assessments for mice bearing subcutaneous xenografts of 143.98.2 osteosarcoma cells.

FIGS. 14C and 14D show tumor growth and survival assessments for mice bearing subcutaneous xenografts of A673 Ewing sarcoma cells.

### DETAILED DESCRIPTION OF THE INVENTION

The present invention provides methods of treating pediatric solid tumors. We have found that a composition comprising nanoparticles comprising a taxane and an albumin, namely, Nab-paclitaxel (Abraxane®), has significant antitumor activity against pediatric solid tumor both in vitro and in vivo. It was further shown that Nab-paclitaxel (Abraxane®) was active in local relapsed tumors in a pediatric solid tumor xenograft model following prior paclitaxel treatment.

Thus, the present application in one aspect provides a method of treating solid tumor in a human individual comprising administering to the individual an effective amount of a composition comprising nanoparticles comprising a taxane and albumin, wherein the individual is no more than about 21 years old (such as no more than about 18 years old). The solid tumor includes, for example, a soft tissue sarcoma (such as rhabdomyosarcoma) and neuroblastoma.

In another aspect, there is provided a method of treating solid tumor in a human individual comprising administering to the individual an effective amount of a composition comprising nanoparticles comprising a taxane and albumin, wherein the individual is no more than about 21 years old (such as no more than about 18 years old), and wherein the individual has had a prior treatment. In some embodiments, the individual is resistant or refractory to the prior treatment. In some embodiments, the individual has progressed on the prior treatment. In some embodiments, the individual has a recurrent solid tumor.

Also provided are compositions (such as pharmaceutical compositions), medicine, kits, and unit dosages useful for the methods described herein.

### DEFINITIONS

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer. The methods of the invention contemplate any one or more of these aspects of treatment.

The term “individual” refers to a mammal and includes, but is not limited to, human, bovine, horse, feline, canine, rodent, or primate.

“Prior therapy” used herein refers to a therapeutic regime that is different from and was instituted prior to the methods of administering the nanoparticle compositions. The prior therapy generally, but not necessarily, does not involve the administration of the taxane nanoparticle composition. It is to be understood that the prior therapy may involve some of the same therapeutic agent(s) with the methods described herein.

As used herein, an “at risk” individual is a human individual who is at risk of developing solid tumor. A human individual “at risk” may or may not have detectable disease, and may or may not have displayed detectable disease prior to the treatment methods described herein. “At risk” denotes that a human individual has one or more so-called risk factors, which are measurable parameters that correlate with development of solid tumor, which are described herein. A human individual having one or more of these risk factors has a higher probability of developing cancer than a human individual without these risk factor(s).

“Adjuvant setting” refers to a clinical setting in which a human individual has had a history of solid tumor, and generally (but not necessarily) been responsive to therapy, which includes, but is not limited to, surgery (e.g., surgery resection), radiotherapy, and chemotherapy. However, because of their history of solid tumor, these individuals are considered at risk of development of the disease. Treatment or administration in the “adjuvant setting” refers to a subsequent mode of treatment. The degree of risk (e.g., when a human individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of solid tumor means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of solid tumor is a method that reduces probability of disease development in a given time frame and/or reduces the extent of the disease in a given time frame, when compared to not using the method. Such comparisons are typically based on clinical studies, using a statistically significant number of subjects. Solid tumor development can be detectable using standard methods, including, but not limited to, computed tomography (CT Scan, e.g., helical spiral CT scan), endoscopic ultrasound (EUS), endoscopic retrograde cholangiopancreatography (ERCP), laparoscopy, or biopsy (e.g., percutaneous needle biopsy or fine needle aspiration). Development may also refer to solid tumor progression that may be initially undetectable and includes recurrence.

As used herein, by “combination therapy” is meant that a first agent be administered in conjunction with another agent. “In conjunction with” refers to administration of one treatment modality in addition to another treatment modality, such as administration of a nanoparticle composition described herein in addition to administration of the other agent to the same individual. As such, “in conjunction with” refers to administration of one treatment modality before, during, or after delivery of the other treatment modality to the individual.

The term “effective amount” used herein refers to an amount of a compound or composition sufficient to treat a specified disorder, condition or disease such as ameliorate, palliate, lessen, and/or delay one or more of its symptoms. In reference to solid tumor, an effective amount comprises an amount sufficient to cause a tumor to shrink and/or to decrease the growth rate of the tumor (such as to suppress tumor growth) or to prevent or delay other unwanted cell proliferation in solid tumor. In some embodiments, an effective amount is an amount sufficient to delay development of solid tumor. In some embodiments, an effective amount is an amount sufficient to prevent or delay recurrence. An effective amount can be administered in one or more administrations. In the case of solid tumor, the effective amount of the drug or composition may: (i) reduce the number of solid tumor cells; (ii) reduce tumor size; (iii) inhibit, retard, slow to some extent and preferably stop solid tumor cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) relieve to some extent one or more of the symptoms associated with solid tumor; and/or (viii) disrupting (such as destroying) solid tumor stroma.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

### Methods of Treating Pediatric Solid Tumor

The present application in some embodiments provides a method of treating a solid tumor in a human individual, comprising administering to the individual an effective amount of a composition comprising nanoparticles comprising a taxane and albumin, wherein the individual is no more than about 21 years old (such as no more than about 18 years old). In some embodiments, the composition comprises nanoparticles comprising a taxane coated with albumin. In some embodiments, the composition comprises nanoparticles having an average diameter of no greater than about 200 nm. In some embodiments, the composition comprises nanoparticles comprising a taxane coated with albumin and have an average diameter of no greater than about 200 nm. In some embodiments, the taxane is paclitaxel. In some embodiments, the composition comprises nanoparticles comprising paclitaxel coated with human albumin, wherein the nanoparticles have an average diameter of no greater than about 150 (such as about 130 nm), wherein the weight ratio of albumin to paclitaxel in the composition is about 9:1 or less (such as about 9:1). In some embodiments, the composition comprises Abraxane (Nab-paclitaxel). In some embodiments, the composition is Abraxane (Nab-paclitaxel). In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the solid tumor is sarcoma. In some embodiments, the solid tumor is carcinoma (such as adenocarcinoma). In some embodiments, the solid tumor is an abdominal tumor, a soft tissue tumor, a bone tumor, or an eye tumor. In some embodiments, the solid tumor is a brain tumor. In some embodiments, the solid tumor is melanoma.

In some embodiments, the solid tumor is a soft tissue sarcoma, such as rhabdomyosarcoma. Thus, for example, in some embodiments, there is provided a method of treating a soft tissue sarcoma in a human individual, comprising administering to the individual an effective amount of a composition comprising nanoparticles comprising a taxane and albumin, wherein the individual is no more than about 21 years old (such as no more than about 18 years old). In some embodiments, there is provided a method of treating rhabdomyosarcoma in a human individual, comprising administering to the individual an effective amount of a composition comprising nanoparticles comprising a taxane and albumin, wherein the individual is no more than about 21 years old (such as no more than about 18 years old). In some embodiments, the composition comprises nanoparticles comprising a taxane coated with albumin. In some embodiments, the composition comprises nanoparticles having an average diameter of no greater than about 200 nm. In some embodiments, the composition comprises nanoparticles comprising a taxane coated with albumin and have an average diameter of no greater than about 200 nm. In some embodiments, the taxane is paclitaxel. In some embodiments, the composition comprises nanoparticles comprising paclitaxel coated with human albumin, wherein the nanoparticles have an average diameter of no greater than about 150 (such as about 130 nm), wherein the weight ratio of albumin to paclitaxel in the composition is about 9:1 or less (such as about 9:1). In some embodiments, the composition comprises Abraxane (Nab-paclitaxel). In some embodiments, the composition is Abraxane (Nab-paclitaxel). In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the solid tumor is neuroblastoma. For example, in some embodiments, there is provided a method of treating neuroblastoma in a human individual, comprising administering to the individual an effective amount of a composition comprising nanoparticles comprising a taxane and albumin, wherein the individual is no more than about 21 years old (such as no more than about 18 years old). In some embodiments, the composition comprises nanoparticles comprising a taxane coated with albumin. In some embodiments, the composition comprises nanoparticles having an average diameter of no greater than about 200 nm. In some embodiments, the composition comprises nanoparticles comprising a taxane coated with albumin and have an average diameter of no greater than about 200 nm. In some embodiments, the taxane is paclitaxel. In some embodiments, the composition comprises nanoparticles comprising paclitaxel coated with human albumin, wherein the nanoparticles have an average diameter of no greater than about 150 (such as about 130 nm), wherein the weight ratio of albumin to paclitaxel in the composition is about 9:1 or less (such as about 9:1). In some embodiments, the composition comprises Abraxane (Nab-paclitaxel). In some embodiments, the composition is Abraxane (Nab-paclitaxel). In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the solid tumor is an early stage solid tumor, such as Stage 0, Stage I, or Stage II. In some embodiments, the solid tumor is a late stage cancer, such as Stage III or Stage IV. In some embodiments, the solid tumor is at stage IIIb or Stage IV.

In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. The methods described herein thus in some embodiments also encompasses selecting a human individual for treatment based on the age of the individual (such as the ages indicated above).

In some embodiments, the solid tumor is early stage cancer, non-metastatic cancer, primary cancer, advanced cancer, locally advanced cancer, metastatic cancer, cancer in remission, or recurrent cancer. In some embodiments, the solid tumor is localized resectable, localized unresectable, or unresectable. In some embodiments, the solid tumor is a progressive solid tumor. In some embodiments, the solid tumor is substantially refractory to hormone therapy. The methods provided herein can be practiced in an adjuvant setting. Alternatively, the methods can be practiced in a neoadjuvant setting. In some embodiments, the method is a first line therapy. In some embodiments, the method is a second line therapy.

In some embodiments, the individual has been previously treated for the solid tumor (also referred to as the “prior therapy”). Thus, for example, in some embodiments, there is provided a method of treating a solid tumor in a human individual, comprising administering to the individual an effective amount of a composition comprising nanoparticles comprising a taxane and albumin, wherein the individual is no more than about 21 years old (such as no more than about 18 years old), and wherein the individual has been previously treated for the solid tumor. In some embodiments, there is provided a method of treating a sarcoma (such as a soft tissue sarcoma, for example rhabdomyosarcoma) in a human individual, comprising administering to the individual an effective amount of a composition comprising nanoparticles comprising a taxane and albumin, wherein the individual is no more than about 21 years old (such as no more than about 18 years old), and wherein the individual has been previously treated for the sarcoma. In some embodiments, there is provided a method of treating neuroblastoma in a human individual, comprising administering to the individual an effective amount of a composition comprising nanoparticles comprising a taxane and albumin, wherein the individual is no more than about 21 years old (such as no more than about 18 years old), and wherein the individual has been previously treated for neuroblastoma. In some embodiments, the composition comprises nanoparticles comprising a taxane coated with albumin. In some embodiments, the composition comprises nanoparticles having an average diameter of no greater than about 200 nm. In some embodiments, the composition comprises nanoparticles comprising a taxane coated with albumin and have an average diameter of no greater than about 200 nm. In some embodiments, the taxane is paclitaxel. In some embodiments, the composition comprises nanoparticles comprising paclitaxel coated with human albumin, wherein the nanoparticles have an average diameter of no greater than about 150 (such as about 130 nm), wherein the weight ratio of albumin to paclitaxel in the composition is about 9:1 or less (such as about 9:1). In some embodiments, the composition comprises Abraxane (Nab-paclitaxel). In some embodiments, the composition is Abraxane (Nab-paclitaxel). In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the individual has progressed on the prior therapy at the time of treatment. For example, the individual has progressed within any of about 1, 2, 3, 4, 5, 6, 7, 8, 9, 10, 11, or 12 months upon treatment with the prior therapy. In some embodiments, the individual is resistant or refractory to the prior therapy. In some embodiments, the individual is unsuitable to continue with the prior therapy (for example due to failure to respond and/or due to toxicity). In some embodiments, the individual has failed to respond to the prior therapy. In some embodiments, the individual is non-responsive to the prior therapy. In some embodiments, the individual is partially responsive to the prior therapy. In some embodiments, the individual exhibits a less desirable degree of responsiveness. In some embodiments, the individual exhibits enhanced responsiveness. In some embodiments, the individual has recurrent solid tumor, i.e., the individual is initially responsive to the treatment with the prior therapy, but develops solid tumor after about any of about 2, 3, 4, 5, 6, 7, 8, 9, 10, 11, 12, 24, 36, 48, or 60 months upon the cessation of the prior therapy.

In some embodiments, the prior therapy has stopped (for example for at least 1, 2, 3, 4, 5, 6, 7, 8, 9, 10, 11, 12, 24, 36, 48, or 60 months) when initiating the methods of the present invention. In some embodiments, the prior therapy has not stopped when initialing the methods of the present invention.

In some embodiments, the method further comprises a step of selecting patients for treatment. For example, in some embodiments, there is provided a method of treating a solid tumor (for example neuroblastoma and soft tissue sarcoma such as rhabdomyosarcoma) in a human individual who has been treated with a prior therapy, the method comprising: a) determining whether the individual has progressed on the prior therapy (such as taxane-based therapy), wherein the individual is no more than about 21 years old (such as no more than about 18 years old), and b) administering an effective amount of a composition comprising nanoparticles comprising albumin and a taxane to the individual. In some embodiments, there is provided a method of treating a solid tumor (for example neuroblastoma and soft tissue sarcoma such as rhabdomyosarcoma) in a human individual who has been treated with a prior therapy, the method comprising: a) selecting the individual who is not responsive to the prior therapy (such as taxane-based therapy), wherein the individual is no more than about 21 years old (such as no more than about 18 years old), and b) administering an effective amount of a composition comprising nanoparticles comprising albumin and a taxane to the individual. In some embodiments, there is provided a method of treating solid tumor (for example neuroblastoma and soft tissue sarcoma such as rhabdomyosarcoma) in a human individual who has been treated with a prior therapy (such as taxane-based therapy), the method comprising administering an effective amount of a composition comprising nanoparticles comprising albumin and a taxane to the individual, wherein the individual is no more than about 21 years old (such as no more than about 18 years old), and wherein said individual is selected for treatment based on the determination that the individual has progressed on the prior therapy. In some embodiments, there is provided a method of treating a solid tumor (for example neuroblastoma and soft tissue sarcoma such as rhabdomyosarcoma) in a human individual who has been treated with a prior therapy (such as taxane-based therapy), the method comprising administering an effective amount of a composition comprising nanoparticles comprising albumin and a taxane to the individual, wherein the individual is no more than about 21 years old (such as no more than about 18 years old), and wherein said individual is selected on the basis of the non-responsiveness to the prior therapy. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of treating a solid tumor (for example neuroblastoma and soft tissue sarcoma such as rhabdomyosarcoma) in a human individual who has been treated with a prior therapy (such as taxane-based therapy), the method comprising: a) determining whether the individual is suitable for continued treatment with the prior therapy (for example due to lack of responsiveness and/or toxicity), wherein the individual is no more than about 21 years old (such as no more than about 18 years old); and b) administering an effective amount of a composition comprising nanoparticles comprising albumin and a taxane to the individual. In some embodiments, there is provided a method of treating a solid tumor (for example neuroblastoma and soft tissue sarcoma such as rhabdomyosarcoma) in a human individual who has been treated with a prior therapy (such as taxane-based therapy), the method comprising administering an effective amount of a composition comprising nanoparticles comprising albumin and a taxane to the individual, wherein the individual is no more than about 21 years old (such as no more than about 18 years old), and wherein said individual is selected based on the determination that the individual is unsuitable for continued treatment with the prior therapy (for example due to lack of responsiveness and/or toxicity). A human individual can also be unsuitable for continued treatment with the prior therapy if the individual exhibits a less than desirable responsiveness or exhibits undesirable symptoms associated with the prior therapy. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of treating a solid tumor (for example neuroblastoma and soft tissue sarcoma such as rhabdomyosarcoma) in a human individual who has been treated with a prior therapy, the method comprising: a) determining whether the individual is resistant or refractory to the prior therapy (such as taxane-based therapy), wherein the individual is no more than about 21 years old (such as no more than about 18 years old); and b) administering an effective amount of a composition comprising nanoparticles comprising albumin and a taxane to the individual. In some embodiments, there is provided a method of treating a solid tumor (for example neuroblastoma and soft tissue sarcoma such as rhabdomyosarcoma) in a human individual who has been treated with a prior therapy, the method comprising administering an effective amount of a composition comprising nanoparticles comprising albumin and a taxane to the individual, wherein the individual is no more than about 21 years old (such as no more than about 18 years old), and wherein said individual is selected based on the determination that the individual is resistant or refractory to the prior therapy (such as taxane-based therapy). In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the prior therapy comprises administration of a taxane (“taxane-based therapy”), such as paclitaxel for example Taxol®. In some embodiments, the prior therapy comprises the administration of Cosmegen (Dactinomycin, also known as actinomycin-D), Vincasar PFS (Vincristine Sulfate), cyclophospha mide, Doxorubicin Hydrochloride (Adriamycin PFS or Adriamycin RDF), carboplatin, cisplatin, etoposide, teniposide, cyclosporin, dacarbazine, epirubicin, gemcitabine, ifosfamide, methotrexate, topotecan, and/or dactinomycin. In some embodiments, the prior therapy comprises surgery.

In some embodiments, the method described herein comprises administering taxane nanoparticle composition in conjunction with one or more of the same agent(s) used in the prior therapy. In some embodiments, the method described herein comprises administering taxane nanoparticle composition in conjunction with the agent(s) that is not used in the prior therapy.

In some embodiments, the method comprises administering to the individual an effective amount of a composition comprising nanoparticles comprising a taxane and an albumin, wherein the individual is no more than about 21 years old (such as no more than about 18 years old), and wherein the individual has progressed on a prior therapy (such as taxane-based therapy). In some embodiments, the method comprises administering to the individual an effective amount of a composition comprising nanoparticles comprising a taxane and an albumin, wherein the taxane is coated with the albumin, wherein the individual is no more than about 21 years old (such as no more than about 18 years old), and wherein the individual has progressed on a prior therapy (such as taxane-based therapy). In some embodiments, there is provided a method of treating a solid tumor in a human individual, comprising administering to the individual an effective amount of a composition comprising nanoparticles comprising a taxane and an albumin, wherein the average particle size of the nanoparticles in the nanoparticle composition is no greater than about 200 nm (such as less than about 200 nm), wherein the individual is no more than about 21 years old (such as no more than about 18 years old), and wherein the individual has progressed on a taxane-based therapy. In some embodiments, there is provided a method of treating a solid tumor in a human individual, comprising administering to the individual an effective amount of a composition comprising nanoparticles comprising a taxane and an albumin, wherein the taxane is coated with the albumin, wherein the average particle size of the nanoparticles in the nanoparticle composition is no greater than about 200 nm (such as less than about 200 nm), wherein the individual is no more than about 21 years old (such as no more than about 18 years old), and wherein the individual has progressed on taxane-based therapy. In some embodiments, there is provided a method of treating a solid tumor in a human individual, comprising administering to the individual an effective amount of a composition comprising nanoparticles comprising paclitaxel and human albumin, wherein the paclitaxel is coated with the human albumin, wherein the average particle size of the nanoparticles in the nanoparticle composition is no greater than about 150 nm (such as about 150 nm), wherein the weight ratio of human albumin and paclitaxel is about 9:1 or less, wherein the individual is no more than about 21 years old (such as no more than about 18 years old), and wherein the individual has progressed on taxane-based therapy. In some embodiments, there is provided a method of treating a solid tumor in a human individual, comprising administering to the individual an effective amount of a composition comprising Nab-paclitaxel, wherein the individual is no more than about 21 years old (such as no more than about 18 years old), and wherein the individual has progressed on taxane-based therapy. In some embodiments, there is provided a method of treating a solid tumor in a human individual, comprising administering to the individual an effective amount of Nab-paclitaxel, wherein the individual is no more than about 21 years old (such as no more than about 18 years old), and wherein the individual has progressed on taxane-based therapy. In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the method comprises administering to the individual an effective amount of a composition comprising nanoparticles comprising a taxane and an albumin, wherein the individual is no more than about 21 years old (such as no more than about 18 years old), and wherein the individual is resistant or refractory to a prior therapy (such as taxane-based therapy). In some embodiments, the method comprises administering to the individual an effective amount of a composition comprising nanoparticles comprising a taxane and an albumin, wherein the taxane is coated with the albumin, wherein the individual is no more than about 21 years old (such as no more than about 18 years old), and wherein the individual is resistant or refractory to a prior therapy (such as taxane-based therapy). In some embodiments, there is provided a method of treating a solid tumor in a human individual, comprising administering to the individual an effective amount of a composition comprising nanoparticles comprising a taxane and an albumin, wherein the average particle size of the nanoparticles in the nanoparticle composition is no greater than about 200 nm (such as less than about 200 nm), wherein the individual is no more than about 21 years old (such as no more than about 18 years old), and wherein the individual is resistant or refractory to a prior therapy (such as taxane-based therapy). In some embodiments, there is provided a method of treating a solid tumor in a human individual, comprising administering to the individual an effective amount of a composition comprising nanoparticles comprising paclitaxel and a human albumin, wherein the taxane is coated with the albumin, wherein the average particle size of the nanoparticles in the nanoparticle composition is no greater than about 150 nm (such as about 150 nm), wherein the weight ratio of human albumin and paclitaxel in the composition is about 9:1 or less (such as about 9:1), wherein the individual is no more than about 21 years old (such as no more than about 18 years old), and wherein the individual is resistant or refractory to a prior therapy (such as taxane-based therapy). In some embodiments, there is provided a method of treating a solid tumor in a human individual, comprising administering to the individual an effective amount of a composition comprising Nab-paclitaxel, wherein the individual is no more than about 21 years old (such as no more than about 18 years old), and wherein the individual is resistant or refractory to a prior therapy (such as taxane-based therapy). In some embodiments, there is provided a method of treating a solid tumor in a human individual, comprising administering to the individual an effective amount of Nab-paclitaxel, wherein the individual is no more than about 21 years old (such as no more than about 18 years old), and wherein the individual is resistant or refractory to a prior therapy (such as taxane-based therapy). In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the method comprises administering to the individual an effective amount of a composition comprising nanoparticles comprising a taxane and an albumin, wherein the individual is no more than about 21 years old (such as no more than about 18 years old), and wherein the individual has failed to respond to a prior therapy (such as taxane-based therapy). In some embodiments, the method comprises administering to the individual an effective amount of a composition comprising nanoparticles comprising a taxane and an albumin, wherein the taxane is coated with the albumin, wherein the individual is no more than about 21 years old (such as no more than about 18 years old), and wherein the individual has failed to respond to a prior therapy (such as taxane-based therapy). In some embodiments, there is provided a method of treating a solid tumor in a human individual, comprising administering to the individual an effective amount of a composition comprising nanoparticles comprising a taxane and an albumin, wherein the average particle size of the nanoparticles in the nanoparticle composition is no greater than about 200 nm (such as less than about 200 nm), wherein the individual is no more than about 21 years old (such as no more than about 18 years old), and wherein the individual has failed to respond to a prior therapy (such as taxane-based therapy). In some embodiments, there is provided a method of treating a solid tumor in a human individual, comprising administering to the individual an effective amount of a composition comprising nanoparticles comprising a taxane and an albumin, wherein the taxane is coated with the albumin, wherein the average particle size of the nanoparticles in the nanoparticle composition is no greater than about 200 nm (such as less than about 200 nm), wherein the individual is no more than about 21 years old (such as no more than about 18 years old), and wherein the individual has failed to respond to a prior therapy (such as taxane-based therapy). In some embodiments, there is provided a method of treating a solid tumor in a human individual, comprising administering to the individual an effective amount of a composition comprising nanoparticles comprising paclitaxel and a human albumin, wherein the paclitaxel is coated with the human albumin, wherein the average particle size of the nanoparticles in the nanoparticle composition is no greater than about 150 nm (such as about 130 nm), wherein the weight ratio of the human albumin and the paclitaxel is about 9:1 or less (such as about 9:1) wherein the individual is no more than about 21 years old (such as no more than about 18 years old), and wherein the individual has failed to respond to a prior therapy (such as taxane-based therapy). In some embodiments, there is provided a method of treating a solid tumor in a human individual, comprising administering to the individual an effective amount of a composition comprising Nab-paclitaxel, wherein the individual is no more than about 21 years old (such as no more than about 18 years old), and wherein the individual has failed to respond to a prior therapy (such as taxane-based therapy). In some embodiments, there is provided a method of treating a solid tumor in a human individual, comprising administering to the individual an effective amount of Nab-paclitaxel, wherein the individual is no more than about 21 years old (such as no more than about 18 years old), and wherein the individual has failed to respond to a prior therapy (such as taxane-based therapy). In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the method comprises administering to the individual an effective amount of a composition comprising nanoparticles comprising a taxane and an albumin, wherein the individual is no more than about 21 years old (such as no more than about 18 years old), and wherein the individual exhibits a less desirable degree of responsiveness to a prior therapy (such as a taxane-based therapy). In some embodiments, the method comprises administering to the individual an effective amount of a composition comprising nanoparticles comprising a taxane and an albumin, wherein the taxane is coated with the albumin, wherein the individual is no more than about 21 years old (such as no more than about 18 years old), and wherein the individual exhibits a less desirable degree of responsiveness to a prior therapy (such as a taxane-based therapy). In some embodiments, there is provided a method of treating a solid tumor in a human individual, comprising administering to the individual an effective amount of a composition comprising nanoparticles comprising a taxane and an albumin, wherein the average particle size of the nanoparticles in the nanoparticle composition is no greater than about 200 nm (such as less than about 200 nm), wherein the individual is no more than about 21 years old (such as no more than about 18 years old), and wherein the individual exhibits a less desirable degree of responsiveness to a prior therapy (such as a taxane-based therapy). In some embodiments, there is provided a method of treating a solid tumor in a human individual, comprising administering to the individual an effective amount of a composition comprising nanoparticles comprising a taxane and an albumin, wherein the taxane is coated with the albumin, wherein the average particle size of the nanoparticles in the nanoparticle composition is no greater than about 200 nm (such as less than about 200 nm), wherein the individual is no more than about 21 years old (such as no more than about 18 years old), and wherein the individual exhibits a less desirable degree of responsiveness to a prior therapy (such as a taxane-based therapy). In some embodiments, there is provided a method of treating a solid tumor in a human individual, comprising administering to the individual an effective amount of a composition comprising nanoparticles comprising paclitaxel and human albumin, wherein the paclitaxel is coated with the human albumin, wherein the average particle size of the nanoparticles in the nanoparticle composition is no greater than about 150 nm (such as about 130 nm), wherein the weight ratio of human albumin and paclitaxel in the composition is about 9:1 or less (such as about 9:1), wherein the individual is no more than about 21 years old (such as no more than about 18 years old), and wherein the individual exhibits a less desirable degree of responsiveness to a prior therapy (such as a taxane-based therapy). In some embodiments, there is provided a method of treating a solid tumor in a human individual, comprising administering to the individual an effective amount of a composition comprising Nab-paclitaxel, wherein the individual is no more than about 21 years old (such as no more than about 18 years old), and wherein the individual exhibits a less desirable degree of responsiveness to a prior therapy (such as a taxane-based therapy). In some embodiments, there is provided a method of treating a solid tumor in a human individual, comprising administering to the individual an effective amount of Nab-paclitaxel, wherein the individual is no more than about 21 years old (such as no more than about 18 years old), and wherein the individual exhibits a less desirable degree of responsiveness to a prior therapy (such as a taxane-based therapy). In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the method comprises administering to the individual an effective amount of a composition comprising nanoparticles comprising a taxane and an albumin, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a prior therapy). In some embodiments, the method comprises administering to the individual an effective amount of a composition comprising nanoparticles comprising a taxane and an albumin, wherein the taxane is coated with the albumin, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a prior therapy). In some embodiments, there is provided a method of treating a solid tumor in a human individual, comprising administering to the individual an effective amount of a composition comprising nanoparticles comprising a taxane and an albumin, wherein the average particle size of the nanoparticles in the nanoparticle composition is no greater than about 200 nm (such as less than about 200 nm), wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a prior therapy). In some embodiments, there is provided a method of treating a solid tumor in a human individual, comprising administering to the individual an effective amount of a composition comprising nanoparticles comprising a taxane and an albumin, wherein the taxane is coated with the albumin, wherein the average particle size of the nanoparticles in the nanoparticle composition is no greater than about 200 nm (such as less than about 200 nm), wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a prior therapy). In some embodiments, there is provided a method of treating a solid tumor in a human individual, comprising administering to the individual an effective amount of a composition comprising nanoparticles comprising paclitaxel and human albumin, wherein the paclitaxel is coated with the human albumin, wherein the average particle size of the nanoparticles in the nanoparticle composition is no greater than about 150 nm (such as about 130 nm), wherein the weight ratio of human albumin and paclitaxel in the composition is about 9:1 or less (such as about 9:1), wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a prior therapy). In some embodiments, there is provided a method of treating solid tumor in a human individual, comprising administering to the individual an effective amount of a composition comprising Nab-paclitaxel, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a prior therapy). In some embodiments, there is provided a method of treating a solid tumor in a human individual, comprising administering to the individual an effective amount of Nab-paclitaxel, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of prior therapy). In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the method comprises administering to the individual an effective amount of a composition comprising nanoparticles comprising a taxane and an albumin, wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, the method comprises administering to the individual an effective amount of a composition comprising nanoparticles comprising a taxane and an albumin, wherein the taxane is coated with the albumin, wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, there is provided a method of treating a solid tumor in a human individual, comprising administering to the individual an effective amount of a composition comprising nanoparticles comprising a taxane and an albumin, wherein the average particle size of the nanoparticles in the nanoparticle composition is no greater than about 200 nm (such as less than about 200 nm), wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, there is provided a method of treating a solid tumor in a human individual, comprising administering to the individual an effective amount of a composition comprising nanoparticles comprising a taxane and an albumin, wherein the taxane is coated with the albumin, wherein the average particle size of the nanoparticles in the nanoparticle composition is no greater than about 200 nm (such as less than about 200 nm), wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, there is provided a method of treating a solid tumor in a human individual, comprising administering to the individual an effective amount of a composition comprising nanoparticles comprising paclitaxel and human albumin, wherein the paclitaxel is coated with the human albumin, wherein the average particle size of the nanoparticles in the nanoparticle composition is no greater than about 200 nm (such as less than about 200 nm), wherein the weight ratio of human albumin and paclitaxel in the composition is about 9:1 or less (such as about 9:1), wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, there is provided a method of treating a solid tumor in a human individual, comprising administering to the individual an effective amount of a composition comprising Nab-paclitaxel, wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, there is provided a method of treating a solid tumor in a human individual, comprising administering to the individual an effective amount of Nab-paclitaxel, wherein the individual is no more than about 21 years old (such as no more than about 18 years old), and wherein a prior therapy (such as a taxane-based therapy) has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, the solid tumor is a neuroendocrine tumor. In some embodiments, the solid tumor is a cancer of the connective tissue. In some embodiments, the solid tumor is a cancer arising from mesenchymal cells (e.g., skeletal muscle progenitor cells). In some embodiments, the solid tumor is a soft tissue tumor (such as soft tissue sarcoma). In some embodiments, the solid tumor is selected from the group consisting of neuroblastoma, rhabdomyosarcoma, osteosarcoma, retinoblastoma, CNS tumor, Wilm's tumor, and Ewing's sarcoma.

In some embodiments, the solid tumor is neuroblastoma. Neuroblastoma is the most common extracranial solid tumor cancer in childhood and the most common cancer in infancy. Neuroblastoma has an incidence rate of about 650 cases per year in the United States. Neuroblastoma is a neuroendocrine tumor that arises from any neural crest element of the sympathetic nervous system. It frequently originates in one of the adrenal glands, but it can also develop in nerve tissues in the head, neck, chest, and abdomen. In Stage 1 neuroblastoma, the tumor is in only one area and all of the tumor that can be seen can be removed during surgery. In Stage 2A, the tumor is in only one area, but all of the tumor that can be seen cannot be removed during surgery. In Stage 2B, the tumor is in only one area, all of the tumor that can be seen may be completely removed during surgery, and cancer cells are found in the lymph nodes near the tumor. In Stage 3, the tumor cannot be completely removed during surgery, has spread from one side of the body to the other, and may have also spread to nearby lymph nodes. In Stage 4, the tumor has spread to distant lymph nodes, the skin, bone marrow, bone, liver, or the other parts of the body. Stage 4S is diagnosed in infants less than 12 months old with localized primary tumor as defined in Stage 1 or 2, with dissemination limited to liver, skin, or bone marrow. Between 20%-50% of high-risk neuroblastoma cases do not respond adequately to induction high-dose chemotherapy and are progressive or refractory. Relapse after completion of frontline therapy is also common. Growth reduction, thyroid function disorders, learning difficulties, and greater risk of secondary cancers affect survivors of high-risk disease.

Thus, in some embodiments, the solid tumor is Stage I neuroblastoma. In some embodiments, the solid tumor is Stage 2A neuroblastoma. In some embodiments, the solid tumor is Stage I neuroblastoma. In some embodiments, the solid tumor is Stage 3 neuroblastoma. In some embodiments, the solid tumor is Stage I neuroblastoma. In some embodiments, the solid tumor is Stage 4S neuroblastoma. In some embodiments, the individual has neuroblastoma and has had a prior therapy (such as a prior high-dose chemotherapy). In some embodiments, the individual has neuroblastoma and has had a prior therapy (such as a prior high-dose chemotherapy) and is progressive or refractory to the prior therapy.

In some embodiments, the solid tumor is rhabdomyo sarcoma. Rhabdomyosarcoma (RMS) is a cancer of the connective tissue that can arise from mesenchymal cells (i.e., skeletal muscle progenitor cells). RMS can also be found attached to muscle tissue, wrapped around intestines, or in any anatomic location. Most RMS occurs in areas naturally lacking in skeletal muscle, such as the head, neck, or genitourinary tract. Its two most common forms are embryonal RMS and alveolar RMS. Embryonal RMA is more common in infants and younger children, and the cancer cells resemble those of a typical 6-to-8-week embryo. Alveolar RMS is more common in older children and teenagers, and the cancer cells resemble those of a 10-to-12-week embryo. Alveolar RMS can occur in the large muscles of the trunk and legs.

In Stage 1 RMS, the tumor has started in a favorable site, e.g., the orbit of the eye, the head and neck area, a genital or urinary site (except the bladder and prostate), or in the bile ducts. A Stage 1 RMS tumor can be any size and may have grown into nearby areas and/or spread to nearby lymph nodes. A Stage 1 RMS tumor has not spread to distant sites. In Stage 2 RMS, the tumor has started in an unfavorable site, e.g., bladder or prostate, arm or leg, a parameningeal site, or any other site listed in Stage 1. The tumor is about 2 inches or smaller across and has not spread to nearby lymph nodes or distant sites. In Stage 3 RMS, the tumor has started in an unfavorable site, and is either ≦2 inches across but has spread to nearby lymph nodes or is ≧2 inches across and may or may not have spread to the lymph nodes. In either case, the cancer has not spread to distant sites. In Stage 4, the cancer can have started at any site and can be of any size, but it has spread to distant sites such as the bone marrow, lungs, liver, bones, or bone marrow.

The prognosis for a child or adolescent with rhabdomyosarcoma is related to, but not limited to, the age of the patient, site of origin, tumor size (widest diameter), resectability, presence of metastases, number of metastatic sites or tissues involved, presence or absence of regional lymph node involvement, histopathologic subtype (alveolar vs. embryonal) as well as unique biological characteristics of rhabdomyosarcoma tumor cells. Rhabdomyosarcoma is usually curable in most children with localized disease, with more than 70% surviving 5 years after diagnosis. Relapses are uncommon after 5 years of disease-free survival, with a 9% late-event rate at 10 years. Relapses, however, are more common for patients who have gross residual disease in unfavorable sites following initial surgery and those who have metastatic disease at diagnosis.

Thus, in some embodiments, the solid tumor is embryonal rhabdomyosarcoma. In some embodiments, the solid tumor is alveolar RMS (for example alveolar in the large muscles of the trunk and/or legs). In some embodiments, the individual has Stage 1 rhabdomyosarcoma. In some embodiments, the individual has Stage 2 rhabdomyosarcoma. In some embodiments, the individual has Stage 3 rhabdomyosarcoma. In some embodiments, the individual has Stage 4 rhabdomyosarcoma. In some embodiments, the individual having rhabdomyosarcoma is about 6 months to about 7 years old, for example about 6 months to about 5 years old. In some embodiments, the individual having rhabdomyosarcoma is about 9 to about 15 years old, for example about 11 to about 15 years old. In some embodiments, the individual has had a prior treatment, and has had a treatment free period for 3, 4, or 5 years or more.

In some embodiments, the solid tumor is osteosarcoma. Osteosarcoma (OS) is a malignant neoplasm arising from primitive transformed cells of mesenchymal origin that exhibit osteoblastic differentiation and produce malignant osteoid (i.e., the unmineralized, organic portion of the bone matrix that forms prior to the maturation of bone tissue). OS is the eighth most common form of childhood cancer, comprising 2.4% of all malignancies in pediatric patients. OS originates more frequently in the growing part of tubular long bones, with 42% occurring in the femur, 19% in the tibia, and 10% in the humerus. 8% of cases occur in the jaw, and another 8% occurs in the pelvis. OS is more prevalent in males than in females, and more prevalent in African-American and Hispanic children than in Caucasian children.

Osteosarcoma can be localized, metastatic, or recurrent. In localized OS, the cancer cells have not spread beyond the bone or nearby tissue win which the cancer began. In metastatic OS, the cancer cells have spread from the tissue of origin to other sites in the body (e.g., lungs, other bones). Recurrent OS refers to cases in which the cancer has recurred after treatment. The OS can come back in the tissues where it was first identified, or it may recur in another part of the body (e.g., the lung). Another way to describe the extent of OS is via the “TNM” system, in which the “T” refer to the size and location of the tumor, the “N” refers to whether the cancer has spread to the lymph nodes, and “M” refers to whether the cancer has metastasized to other parts of the body (Ritter et al. (2010) “Osteosarcoma.” Ann Oncol. 21: vii320-vii325).

With treatment, the 5-year survival rates for patients with localized osteosarcoma can be in the range of 60%-80%. OS is more likely to be cures if the tumor is resectable. If metastases are present when the osteosarcoma is first diagnosed, the 5-year survival rate can be in the range or about 15%-30%. The survival rate can be higher if the cancer has spread only to the lungs or if all the tumors can be resected. Other factors that have been linked with an improved prognosis include, but are not limited to, age (younger), sex (female), tumor on arm or leg, tumor(s) being completely resectable, normal blood alkaline phosphatase and LDH levels, and good response to chemotherapy.

In some embodiments, the osteosarcoma is localized. In some embodiments, the osteosarcoma is resectable. In some embodiments, the osteosarcoma is metastatic. In some embodiments, the osteosarcoma is recurrent. In some embodiments, the individual has TX, T0, T1, T2, or T3 osteosarcoma. In some embodiments, the individual has NX, N0, or N1 osteosarcoma. In some embodiments, the individual has MX, M0, M1, M1a, or M1b osteosarcoma. In some embodiments, the individual has GX, G1, G2, G3, or G4 osteosarcoma. In some embodiments, the individual has Stage IA osteosarcoma (T1, N0, M0, G1-G2). In some embodiments, the individual has Stage IB osteosarcoma (T2, N0, M0, G1-G2). In some embodiments, the individual has Stage IIA osteosarcoma (T1, N0, M0, G3-G4). In some embodiments, the individual has Stage IIB osteosarcoma (T2, No, M0, G3-G4). In some embodiments, the individual has Stage III osteosarcoma (T3, N0, M0, any G). In some embodiments, the individual has Stage IVA osteosarcoma (any T, N0, M1a, any G). In some embodiments, the individual has Stage IVB (any T, N1, any M; or any T, any N, M1b, any G). In some embodiments, the individual having the osteosarcoma is a male. In some embodiments, the individual having the osteosarcoma is an African-American or Hispanic individual.

In some embodiments, the solid tumor is retinoblastoma. Retinoblastoma develops in the retina, the light-detecting tissue of the eye. Retinoblastoma is rare and affects approximately 1 in 15,000 live births, but it is the most common inherited childhood malignancy. There are two forms of the disease, a heritable (in which a mutation RB 1 gene is genetically inherited) form and a non-heritable form (which occurs when both copies of the RB 1 gene are mutated after conception). In about two-thirds of cases, only one eye is affected; in the other third, tumors develop in both eyes. The Reese-Ellsworth staging system divides intraocular retinoblastoma into 5 groups. Group 1A, includes patient with one tumor that is smaller than 4 optic disc diameters (DD) and is at or behind the equator of the eye (i.e., where the equator of the divides the front and back halves of the eyeball). In Group 1B, patients have multiple tumors smaller than 4 DD, and all are at or behind the equator. Group 2A patients have one tumor, 4 to 10 DD, at or behind the equator, and in Group 2B, patients have multiple tumors, 4 to 10 DD, at or behind the equator. Group 3A patients have a tumor in front of the equator, and Group 3B patients have one tumor, larger than 10DD, behind the equator. Group 4A patients have multiple tumors, some larger than 10DD, and Group 4B patients have one or more tumors that extend toward the front of the eye to the front edge of the retina. Group 5A patients have tumors involving more than half the retina, and Group 5B patients have vitreous seeding, i.e., spread of tumors into the gelatinous material that fills the eye.

In the developed world, retinoblastoma has one of the best cure rates of all childhood cancers (95-98%), with more than nine out of every ten sufferers surviving into adulthood. The priority is to preserve the life of the child, then to preserve vision, and then to minimize the complications or side effects of treatment. Prognosis depends on the extent of the disease, the size and location of the tumor(s), presence or absence of metastasis, and the tumor's response to therapy. A Group 1 retinoblastoma can be controlled with treatments such as chemotherapy, photocoagulation, cryotherapy, brachytherapy, or external beam radiation. A Group 4 or 5 retinoblastoma can be less responsive to such treatments.

In some embodiments, the solid tumor is a heritable retinoblastoma. In some embodiments, the solid tumor is a non-heritable retinoblastoma. In some embodiments, the solid tumor is Group 1A retinoblastoma. In some embodiments, the solid tumor is Group 1B retinoblastoma. In some embodiments, the solid tumor is Group 2A retinoblastoma. In some embodiments, the solid tumor is Group 2B retinoblastoma. In some embodiments, the solid tumor is Group 3A retinoblastoma. In some embodiments, the solid tumor is Group 3B retinoblastoma. In some embodiments, the solid tumor is Group 4A retinoblastoma. In some embodiments, the solid tumor is Group 4B retinoblastoma. In some embodiments, the solid tumor is Group 5A retinoblastoma. In some embodiments, the solid tumor is Group 5B retinoblastoma.

In some embodiments, the individual has a central nervous system (CNS) tumor, such as an astrocytoma, a brain stem glioma, an ependymoma, a germ cell tumor, or a medulloblastoma. Childhood central nervous system tumors do not typically spread outside the brain and spinal cord. In some embodiments, the CNS tumor is a recurrent CNS tumor.

In some embodiments, the individual has Wilms' tumor (also known as nephroblastoma). In some embodiments, the individual has Stage I Wilms' tumor. In some embodiments, the individual has Stage II Wilms' tumor. In some embodiments, the individual has Stage III Wilms' tumor. In some embodiments, the individual has Stage IV Wilms' tumor. In some embodiments, the individual has Stage V Wilms' tumor. In some embodiments, the individual has recurrent Wilms' tumor.

In some embodiments, the individual has soft tissue sarcoma. In some embodiments, the individual has Stage I soft tissue sarcoma. In some embodiments, the individual has Stage II soft tissue sarcoma. In some embodiments, the individual has Stage III soft tissue sarcoma. In some embodiments, the individual has Stage IV soft tissue sarcoma. In some embodiments, the individual has recurrent soft tissue sarcoma.

In some embodiments, the individual has Ewing's sarcoma. In some embodiments, the individual has localized Ewing's sarcoma. In some embodiments, the individual has metastatic Ewing's sarcoma. In some embodiments, the individual has Stage 1 Ewing's sarcoma. In some embodiments, the individual has Stage 2 Ewing's sarcoma. In some embodiments, the individual has Stage 3 Ewing's sarcoma. In some embodiments, the individual has Stage 4 Ewing's sarcoma. In some embodiments, the individual has recurrent Ewing's sarcoma.

In some embodiments, the individual is resistant to treatment of solid tumor with taxane-based therapy (e.g., taxane monotherapy or combination therapy) and has progressed after treatment (e.g., the solid tumor has been refractory). In some embodiments, the individual is initially responsive to treatment of solid tumor with taxane-based therapy (e.g., taxane monotherapy or combination therapy) but has progressed after treatment. In some embodiments, the individual is human. In some embodiments, the individual has a family history of solid tumor (e.g., at least 2 first-degree relatives affected with solid tumor without accumulation of other cancers or familial diseases). In some embodiments, the individual has one or more hereditary pediatric solid tumor symptoms. For neuroblastoma, symptoms can depend on the location of the primary tumor. Symptoms of neuroblastoma can include, but are not limited to, e.g., bulging eyes, dark circles around eyes, bone pain, swollen stomach, fatigue, painless, constipation, anemia, bluish lumps under the skin in infants, weakness or paralysis, edema, and lump in the abdomen, neck, or chest. For retinoblastoma, symptoms can include, but are not limited to, e.g., crossed eyes, double vision, visual disturbances, strabismus, eye pain and redness, and differing iris colors in each eye. For osteosarcoma, symptoms include, hut are not limited to, e.g., bone pain than may become worse during exercise or at might, joint tenderness or inflammation, bone fractures due to bone weakness, limited range of motion, fatigue and anemia. For rhabdomyosarcoma, symptoms can range widely depending on the location of the tumor. Such symptoms can include, but are not limited to, e.g., nosebleed, symptoms similar to a sinus infection, earaches, discharge from the ear canal, bulged or crossed eyes, difficult urination, bleeding from the vagina, mass growing from the vagina or around the testicles, abdominal pain and vomiting, and mass or lump in the arm or leg. In some embodiments, the individual is a male. In some embodiments, the individual is a female. In some embodiments, the individual has a single lesion at presentation. In some embodiments, the individual has multiple lesions at presentation.

In some embodiments, the individual is a human who exhibits one or more symptoms associated with a solid tumor. In some embodiments, the individual is at an early stage of solid tumor. In some embodiments, the individual is at an advanced stage of solid tumor. In some embodiments, the individual has non-metastatic solid tumor. In some embodiments, the individual has primary solid tumor. In some of embodiments, the individual is genetically or otherwise predisposed (e.g., having a risk factor) to developing solid tumor. These risk factors include, but are not limited to, age, sex, race, diet, genetic considerations, family history, inherited conditions (e.g., Li-Fraumeni syndrome, neurofibromatosis type 1, Beckwith-Widemann syndrome, Rothmund-Thompson syndrome, Bloom syndrome, Werner syndrome, Costello syndrome, Noonan syndrome), certain diseases (e.g., Paget disease, bone disease), prenatal exposure (e.g., to tobacco or certain medications) and environmental exposure (e.g., to ionizing radiation).

The methods described herein are useful for various aspects of solid tumor treatment as discussed below. These methods in some embodiments further comprise administering to the individual an effective amount of gemcitabine.

In some embodiments, there is provided a method of inhibiting solid tumor cell proliferation in a human individual, comprising administering to the individual an effective amount of a composition comprising nanoparticles comprising a taxane and an albumin, wherein the individual is no more than about 21 years old (such as no more than about 18 years old). In some embodiments, at least about 10% (including for example at least about any of 20%, 30%, 40%, 60%, 70%, 80%, 90%, or 100%) cell proliferation is inhibited. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of inhibiting solid tumor metastasis in a human individual, comprising administering to the individual an effective amount of a composition comprising nanoparticles comprising a taxane and an albumin, wherein the individual is no more than about 21 years old (such as no more than about 18 years old). In some embodiments, at least about 10% (including for example at least about any of 20%, 30%, 40%, 60%, 70%, 80%, 90%, or 100%) metastasis is inhibited. In some embodiments, a method of inhibiting metastasis to one or more lymph nodes is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of inhibiting solid tumor metastasis in a human individual, comprising administering to the individual an effective amount of a composition comprising nanoparticles comprising a taxane and an albumin, wherein the individual is no more than about 21 years old (such as no more than about 18 years old), and wherein the individual is resistant or refractory to a taxane-based therapy. In some embodiments, at least about 10% (including for example at least about any of 20%, 30%, 40%, 60%, 70%, 80%, 90%, or 100%) metastasis is inhibited. In some embodiments, a method of inhibiting metastasis to one or more lymph nodes is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of inhibiting solid tumor metastasis in a human individual, comprising administering to the individual an effective amount of a composition comprising nanoparticles comprising a taxane and an albumin, wherein the individual is no more than about 21 years old (such as no more than about 18 years old), and wherein the individual has failed to respond to a taxane-based therapy. In some embodiments, at least about 10% (including for example at least about any of 20%, 30%, 40%, 60%, 70%, 80%, 90%, or 100%) metastasis is inhibited. In some embodiments, a method of inhibiting metastasis to one or more lymph nodes is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of inhibiting solid tumor metastasis in a human individual, comprising administering to the individual an effective amount of a composition comprising nanoparticles comprising a taxane and an albumin, wherein the individual is no more than about 21 years old (such as no more than about 18 years old), and wherein the individual exhibits a less desirable degree of responsiveness to a taxane-based therapy. In some embodiments, at least about 10% (including for example at least about any of 20%, 30%, 40%, 60%, 70%, 80%, 90%, or 100%) metastasis is inhibited. In some embodiments, a method of inhibiting metastasis to one or more lymph nodes is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of inhibiting solid tumor metastasis in a human individual, comprising administering to the individual an effective amount of a composition comprising nanoparticles comprising a taxane and an albumin, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a taxane-based therapy). In some embodiments, at least about 10% (including for example at least about any of 20%, 30%, 40%, 60%, 70%, 80%, 90%, or 100%) metastasis is inhibited. In some embodiments, a method of inhibiting metastasis to one or more lymph nodes is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of inhibiting solid tumor metastasis in a human individual, comprising administering to the individual an effective amount of a composition comprising nanoparticles comprising a taxane and an albumin, wherein the individual is no more than about 21 years old (such as no more than about 18 years old), and wherein a taxane-based therapy has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, at least about 10% (including for example at least about any of 20%, 30%, 40%, 60%, 70%, 80%, 90%, or 100%) metastasis is inhibited. In some embodiments, a method of inhibiting metastasis to one or more lymph nodes is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing (such as eradicating) pre-existing tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old). In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing (such as eradicating) pre-existing tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual is resistant or refractory to a taxane-based therapy. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing (such as eradicating) pre-existing tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual has failed to respond to a taxane-based therapy. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing (such as eradicating) pre-existing tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual exhibits a less desirable degree of responsiveness to a taxane-based therapy. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing (such as eradicating) pre-existing tumor metastasis (such as metastasis to the lymph node) in a human individual, comprising administering to the individual an effective amount of a composition comprising nanoparticles comprising a taxane and an albumin, and wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a taxane-based therapy). In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing (such as eradicating) pre-existing tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein a taxane-based therapy has stopped (for example for at least 1, 2, 3, 4, 5, 6, 7, 8, 9, or 10 months) when initiating the administration of the effective amount of the composition comprising nanoparticles comprising a taxane and an albumin to the individual. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing incidence or burden of preexisting tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing incidence or burden of preexisting tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual is resistant or refractory to a taxane-based therapy. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing incidence or burden of preexisting tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual has failed to respond to a taxane-based therapy. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing incidence or burden of preexisting tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual exhibits a less desirable degree of responsiveness to a taxane-based therapy. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing incidence or burden of preexisting tumor metastasis (such as metastasis to the lymph node) in a human individual, comprising administering to the individual an effective amount of a composition comprising nanoparticles comprising a taxane and an albumin, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a taxane-based therapy). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing incidence or burden of preexisting solid tumor metastasis (such as metastasis to the lymph node) in a human individual, comprising administering to the individual an effective amount of a composition comprising nanoparticles comprising a taxane and an albumin, and wherein a taxane-based therapy has stopped (for example, for at least 1, 2, 3, 4, 5, 6, 7, 8, 9, or 10 months) when initiating the administration of the effective amount of the composition comprising nanoparticles comprising a taxane and an albumin to the individual. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing solid tumor size in a human individual, comprising administering to the individual an effective amount of a composition comprising nanoparticles comprising a taxane and an albumin, wherein the individual is no more than about 21 years old (such as no more than about 18 years old). In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing tumor size in a human individual, comprising administering to the individual an effective amount of a composition comprising nanoparticles comprising a taxane and an albumin, wherein the individual is no more than about 21 years old (such as no more than about 18 years old), and wherein the individual is resistant or refractory to a taxane-based therapy. In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing solid tumor size in a human individual, comprising administering to the individual an effective amount of a composition comprising nanoparticles comprising a taxane and an albumin, wherein the individual is no more than about 21 years old (such as no more than about 18 years old), and wherein the individual has failed to respond to a taxane-based therapy. In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing solid tumor size in a human individual, comprising administering to the individual an effective amount of a composition comprising nanoparticles comprising a taxane and an albumin, wherein the individual is no more than about 21 years old (such as no more than about 18 years old), and wherein the individual exhibits a less desirable degree of responsiveness to a taxane-based therapy. In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing solid tumor size in a human individual, comprising administering to the individual an effective amount of a composition comprising nanoparticles comprising a taxane and an albumin, wherein the individual is no more than about 21 years old (such as no more than about 18 years old), and wherein the individual has recurrent solid tumor (for example, the individual develops solid tumor after about any of about 2, 3, 4, 5, 6, 7, 8, 9, 10, 11, 12, 24, 36, 48, or 60 months upon the cessation of a taxane-based therapy). In some embodiments, the tumor size is reduced at least about 10% including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of reducing solid tumor size in a human individual, comprising administering to the individual an effective amount of a composition comprising nanoparticles comprising a taxane and an albumin, wherein the individual is no more than about 21 years old (such as no more than about 18 years old), and wherein a taxane-based therapy has stopped (for example for at least 1, 2, 3, 4, 5, 6, 7, 8, 9, or 10 months) when initiating the administration of the effective amount of the composition comprising nanoparticles comprising a taxane and an albumin to the individual. In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of prolonging time to disease progression of solid tumor (e.g., progression-free survival) in a human individual, comprising administering to the individual an effective amount of a composition comprising nanoparticles comprising a taxane and an albumin, wherein the individual is no more than about 21 years old (such as no more than about 18 years old). In some embodiments, the method prolongs the time to disease progression by at least any of 1, 2, 3, 4, 5, 6, 7, 8, 9, 10, 11, 12 weeks. In some embodiments, the method prolongs the time to disease progression by at least any of 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 13, 14, 15, 16, 17, 18, 19, 20, 21, 22, 23, 24, 30, 36, 42, 48, 54, 60, 66, or 72 months. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of prolonging overall survival of a human individual having solid tumor, comprising administering to the individual an effective amount of a composition comprising nanoparticles comprising a taxane and an albumin, wherein the individual is no more than about 21 years old (such as no more than about 18 years old). In some embodiments, the method prolongs the survival of the individual by at least any of 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 13, 14, 15, 16, 17, 18, 19, 20, 21, 22, 23, 24, 30, 36, 42, 48, 54, 60, 66, or 72 months. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of improving one or more clinical benefits of a human individual having a solid tumor, comprising administering to the individual an effective amount of a composition comprising nanoparticles comprising a taxane and an albumin, wherein the individual is no more than about 21 years old (such as no more than about 18 years old). Clinical benefits includes, but are not limited to, improved/better quality of life, improved/better symptom control of the solid tumor, and increased weight gain. In some embodiments, the individual has improved quality of life, improved symptom control and increased weight gain. In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma.

In some embodiments, there is provided a method of alleviating one or more symptoms in a human individual having a solid tumor, comprising administering to the individual an effective amount of a composition comprising nanoparticles comprising a taxane and an albumin, wherein the individual is no more than about 21 years old (such as no more than about 18 years old). In some embodiments, the taxane is paclitaxel. In some embodiments, the taxane in the nanoparticle in the composition is administered by intravenous administration. In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old.

In some embodiments, there is provided a method of treating a solid tumor in a human individual comprising administering to the individual an effective amount of a composition comprising ABRAXANE®, wherein the individual is no more than about 21 years old (such as no more than about 18 years old), and wherein the ABRAXANE® is administered weekly or weekly for three out of four weeks at a dose ranging from about 80 mg/m² to about 150 mg/m² (for example, about 100 mg/m² to about 150 mg/m², e.g., about 100 mg/m²). In some embodiments, there is provided a method of treating a solid tumor in a human individual comprising administering to the individual an effective amount of a composition comprising ABRAXANE®, wherein the individual is no more than about 21 years old (such as no more than about 18 years old), and wherein the ABRAXANE® is administered once every three weeks at a dose ranging from about 150 mg/m² to about 300 mg/m² (for example, about 260 mg/m²). In some embodiments, the ABRAXANE® is administered by intravenous administration. In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine, such as about 750 mg/m² to about 3000 mg/m², including for example about 1000 mg/m² to about 2000 mg/m². In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of treating solid tumor in a human individual comprising administering to the individual an effective amount of a composition comprising ABRAXANE®, wherein the ABRAXANE® is administered weekly or weekly for three out of four weeks at a dose ranging from about 80 mg/m² to about 150 mg/m² (for example, about 100 mg/m² to about 150 mg/m², e.g., about 100 mg/m²), wherein the individual is no more than about 21 years old (such as no more than about 18 years old), and wherein the individual is resistant or refractory to a prior therapy (such as a taxane-based therapy). In some embodiments, there is provided a method of treating solid tumor in a human individual comprising administering to the individual an effective amount of a composition comprising ABRAXANE®, wherein the ABRAXANE® is administered once every three weeks at a dose ranging from about 150 m g/m² to about 300 mg/m² (for example, about 260 mg/m²), wherein the individual is no more than about 21 years old (such as no more than about 18 years old), and wherein the individual is resistant or refractory to a prior therapy (such as a taxane-based therapy). In some embodiments, the ABRAXANE® is administered by intravenous administration. In some embodiments, the individual has non-metastatic solid tumor. In some embodiments, the individual has primary solid tumor. In some embodiments, the individual has sarcoma, such as soft tissue sarcoma, for example rhabdomyosarcoma. In some embodiments, the individual has neuroblastoma. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine, such as about 750 mg/m2 to about 3000 mg/m2, including for example about 1000 mg/m2 to about 2000 mg/m2. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of treating Ewing's sarcoma in a human individual comprising administering to the individual an effective amount of a composition comprising ABRAXANE®, wherein the individual is no more than about 21 years old (such as no more than about 18 years old), and wherein the ABRAXANE® is administered weekly or weekly for three out of four weeks at a dose ranging from about 80 mg/m2 to about 150 mg/m2 (for example, about 100 mg/m2 to about 150 mg/m2, e.g., about 100 mg/m2). In some embodiments, there is provided a method of treating Ewing's sarcoma in a human individual comprising administering to the individual an effective amount of a composition comprising ABRAXANE®, wherein the individual is no more than about 21 years old (such as no more than about 18 years old), and wherein the ABRAXANE® is administered once every three weeks at a dose ranging from about 150 mg/m2 to about 300 mg/m2 (for example, about 260 mg/m2). In some embodiments, the ABRAXANE® is administered by intravenous administration. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine, such as about 750 mg/m2 to about 3000 mg/m2, including for example about 1000 mg/m2 to about 2000 mg/m2. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of prolonging survival of a human individual having Ewing's sarcoma comprising administering to the individual an effective amount of a composition comprising ABRAXANE®, wherein the individual is no more than about 21 years old (such as no more than about 18 years old), and wherein the ABRAXANE® is administered weekly or weekly for three out of four weeks at a dose ranging from about 80 mg/m2 to about 150 mg/m2 (for example, about 100 mg/m2 to about 150 mg/m2, e.g., about 100 mg/m2). In some embodiments, there is provided a method of prolonging survival of a human individual having Ewing's sarcoma comprising administering to the individual an effective amount of a composition comprising ABRAXANE®, wherein the individual is no more than about 21 years old (such as no more than about 18 years old), and wherein the ABRAXANE® is administered once every three weeks at a dose ranging from about 150 mg/m2 to about 300 mg/m2 (for example, about 260 mg/m2). In some embodiments, the ABRAXANE® is administered by intravenous administration. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine, such as about 750 mg/m2 to about 3000 mg/m2, including for example about 1000 mg/m2 to about 2000 mg/m2. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of treating osteosarcoma in a human individual comprising administering to the individual an effective amount of a composition comprising ABRAXANE®, wherein the ABRAXANE® is administered weekly or weekly for three out of four weeks at a dose ranging from about 80 mg/m2 to about 150 mg/m2 (for example, about 100 mg/m2 to about 150 mg/m2, e.g., about 100 mg/m2), wherein the individual is no more than about 21 years old (such as no more than about 18 years old), and wherein the individual is resistant or refractory to a prior therapy (such as a taxane-based therapy). In some embodiments, there is provided a method of treating osteosarcoma in a human individual comprising administering to the individual an effective amount of a composition comprising ABRAXANE®, wherein the ABRAXANE® is administered once every three weeks at a dose ranging from about 150 mg/m2 to about 300 mg/m2 (for example, about 260 mg/m2), wherein the individual is no more than about 21 years old (such as no more than about 18 years old), and wherein the individual is resistant or refractory to a prior therapy (such as a taxane-based therapy). In some embodiments, the ABRAXANE® is administered by intravenous administration. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine, such as about 750 mg/m2 to about 3000 mg/m2, including for example about 1000 mg/m2 to about 2000 mg/m2. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

In some embodiments, there is provided a method of prolonging survival of a human individual having osteosarcoma comprising administering to the individual an effective amount of a composition comprising ABRAXANE®, wherein the ABRAXANE® is administered weekly or weekly for three out of four weeks at a dose ranging from about 80 mg/m2 to about 150 mg/m2 (for example, about 100 mg/m2 to about 150 mg/m2, e.g., about 100 mg/m2), wherein the individual is no more than about 21 years old (such as no more than about 18 years old), and wherein the individual is resistant or refractory to a prior therapy (such as a taxane-based therapy). In some embodiments, there is provided a method of prolonging survival of a human individual having osteosarcoma comprising administering to the individual an effective amount of a composition comprising ABRAXANE®, wherein the ABRAXANE® is administered once every three weeks at a dose ranging from about 150 mg/m2 to about 300 mg/m2 (for example, about 260 mg/m2), wherein the individual is no more than about 21 years old (such as no more than about 18 years old), and wherein the individual is resistant or refractory to a prior therapy (such as a taxane-based therapy). In some embodiments, the ABRAXANE® is administered by intravenous administration. In some embodiments, the individual is no more than about any of 17, 16, 15, 14, 13, 12, 11, 10, 9, 8, 7, 6, 5, 4, 3, 2, or 1 year old. In some embodiments, the individual is about 9 to about 15 years old. In some embodiments, the individual is about 5 to about 9 years old. In some embodiments, the individual is about 1 to about 5 years old. In some embodiments, the individual is no more than about 1 year old, such as about 6 months old to about 1 year old, less than about 6 months old, or less than about 3 months old. In some embodiments, the method further comprises administering (such as intravenously administering) to the individual an effective amount of gemcitabine, such as about 750 mg/m2 to about 3000 mg/m2, including for example about 1000 mg/m2 to about 2000 mg/m2. In some embodiments, the gemcitabine and the nanoparticle composition are administered sequentially. In some embodiments, the gemcitabine and the nanoparticle composition are administered simultaneously. In some embodiments, the gemcitabine and the nanoparticle composition are administered concurrently.

### Dosing and Method of Administering the Nanoparticle Compositions

The dose of the taxane nanoparticle compositions administered to a human individual (such as a human) may vary with the particular composition, the mode of administration, and the type of solid tumor being treated. In some embodiments, the amount of the composition is effective to result in an objective response (such as a partial response, a complete response, or stable disease). In some embodiments, the amount of the taxane nanoparticle composition is sufficient to result in a complete response in the individual. In some embodiments, the amount of the taxane nanoparticle composition is sufficient to result in a partial response in the individual. In some embodiments, the amount of the taxane nanoparticle composition is sufficient to result in stable disease (i.e., solid tumor) in the individual. In some embodiments, the amount of the taxane nanoparticle composition administered (for example when administered alone) is sufficient to produce an overall response rate of more than about any of 25%, 30%, 32%, 35%, 36%, 37%, 38%, 39%, 40%, 50%, 60%, 65%, or 70% among a population of individuals treated with the taxane nanoparticle composition. Responses of a human individual to the treatment of the methods described herein can be determined, for example, based on RECIST levels.

In some embodiments, the amount of the composition is sufficient to prolong progression-free survival of the individual. In some embodiments, the amount of the composition is sufficient to prolong overall survival of the individual. In some embodiments, the amount of the composition (for example when administered along) is sufficient to produce clinical benefits of more than about any of 25%, 30%, 32%, 35%, 36%, 37%, 38%, 39%, 40%, 50%, 60%, 65%, or 70% among a population of individuals treated with the taxane nanoparticle composition.

In some embodiments, the amount of the composition, first therapy, second therapy, or combination therapy is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of solid tumor cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the taxane (e.g., paclitaxel) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regime. In some embodiments, the amount of the composition is more than about any of 80%, 90%, 95%, or 98% of the MTD.

In some embodiments, the amount of a taxane (e.g., paclitaxel) in the composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of a taxane (e.g., paclitaxel) in the dose of the composition (e.g., a unit dosage form) is in the range of about 5 mg to about 500 mg, such as about 30 mg to about 300 mg or about 50 mg to about 200 mg. In some embodiments, the concentration of the taxane (e.g., paclitaxel) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the taxane (e.g., paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary doses of a taxane (e.g., paclitaxel) in the nanoparticle composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m, 540 mg/m², 750 mg/m², 1000 mg/m², or 1080 mg/m² of a taxane (e.g., paclitaxel). In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a taxane (e.g., paclitaxel). In some embodiments, the amount of the taxane (e.g., paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the dose of a taxane paclitaxel) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the dose of a taxane (e.g., paclitaxel) in the composition is about 5 to about 300 mg/m², such as about 100 to about 150 mg/m², about 120 mg/m², about 130 mg/m², or about 140 mg/m². In some embodiments, the dose of a taxane (e.g., paclitaxel) in the composition is about 100 mg/m².

In some embodiments of any of the above aspects, the dose of a taxane paclitaxel) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the dose of a taxane (e.g., paclitaxel) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a taxane (e.g., paclitaxel).

In some embodiments, the dose of paclitaxel in the composition is at least about any of 2 mg/kg, 2.5 mg/kg, 2.7 mg/kg, 5 mg/kg, 6.5 mg/kg, 7.5 mg/kg, or 10 mg/kg administered on days 1, 8, and 15 on a 28-day cycle. In some embodiments, the dose of paclitaxel in the composition is about 2.7 mg/kg administered on days 1, 8, and 15 on a 28-day cycle. In some embodiments, the composition is administered intravenously over 30 minutes.

Exemplary dosing frequencies for the administration of the nanoparticle compositions include, but are not limited to, daily, every two days, every three days, every four days, every five days, every six days, weekly without break, three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 28 days, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the taxane (e.g., paclitaxel) is administered over a period of at least ten days, wherein the interval between each administration is no more than about two days, and wherein the dose of the taxane (e.g., paclitaxel) at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

In some embodiments, the taxane (e.g., paclitaxel) is administered on days 1, 8, and 15 on a 28-day cycle, wherein the dose of the taxane (e.g., paclitaxel) at each administration is about 100 mg/m², mg/m², 150 mg/m², 175 mg/m², or 200 mg/m². In some embodiments, the taxane (e.g., paclitaxel) is administered intravenously over 30 minutes on days 1, 8, and 15 on a 28-day cycle, wherein the dose of the taxane (e.g., paclitaxel) at each administration is about 100 mg/m², 125 mg/m², 150 mg/m², 175 mg/m², or 200 mg/m². In some embodiments, the taxane is paclitaxel.

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months.

In some embodiments, the dosage of a taxane (e.g., paclitaxel) in a nanoparticle composition can be in the range of 5-400 mg/m when given on a 3 week schedule, or 5-250 mg/m² (such as 80-150 mg/m², for example 100-120 mg/m²) when given on a weekly schedule. For example, the amount of a taxane (e.g., paclitaxel) is about 60 to about 300 mg/m² (e.g., about 260 mg/m²) on a four week schedule.

Other exemplary dosing schedules for the administration of the nanoparticle composition (e.g., paclitaxel/albumin nanoparticle composition) include, but are not limited to, 100 mg/m², weekly, without break; 75 mg/m² weekly, 3 out of 4 weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the composition may be adjusted over the course of the treatment based on the judgment of the administering physician.

In some embodiments, the individual is treated for at least about any of one, two, three, four, five, six, seven, eight, nine, or ten treatment cycles.

The compositions described herein allow infusion of the composition to a human individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes, or about 30-40 minutes.

Other exemplary doses of the taxane (in some embodiments paclitaxel) in the nanoparticle composition include, but are not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of paclitaxel in a nanoparticle composition can be in the range of about 100-400 mg/m² when given on a 3 week schedule, or about 50-250 mg/m² when given on a weekly schedule.

The nanoparticle compositions can be administered to a human individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered intraarterially. In some embodiments, the composition is administered intraperitoneally.

When in combination therapy (such as combination therapy with gemcitabine), the other agent (such as gemcitabine) can be administered with the same or different route as the nanoparticle composition. The dosing frequency for administering the other agent can be the same or different from that of the nanoparticle composition. In some embodiments when the other agent is gemcitabine, the gemcitabine can be administered at the dosage of about 500 to about 3000 mg/m², such as about 500 to about 750, about 750 to about 1000, about 1000 to about 1250, about 1250 to about 1500, about 1500 to about 1750, about 1750 to about 2000, about 2000 to about 2250, about 2250 to about 2500, about 2500 to about 2750, or about 2750 to about 3000 mg/m². In some embodiments, the gemcitabine is administered sequentially with the nanoparticle composition. In some embodiments, the gemcitabine is administered simultaneously with the nanoparticle composition. In some embodiments, the gemcitabine is administered concurrently with the nanoparticle composition.

### Nanoparticle Compositions

The nanoparticle compositions described herein comprise nanoparticles comprising (in various embodiments consisting essentially of) a taxane (such as paclitaxel) and an albumin (such as human serum albumin). Nanoparticles of poorly water soluble drugs (such as taxane) have been disclosed in, for example, U.S. Pat. Nos. 5,916,596; 6,506,405; 6,749,868, and 6,537,579; 7,820,788, and US Pat. Pub. Nos., 2006/0263434, and 2007/0082838; PCT Patent Application WO08/137148, each of which is incorporated by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some, embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprise the taxane (such as paclitaxel) coated with an albumin (e.g., human serum albumin). In some embodiments, the composition comprises taxane in both nanoparticle and non-nanoparticle forms, wherein at least about any one of 50%, 60%, 70%, 80%, 90%, 95%, or 99% of the taxane in the composition are in nanoparticle form. In some embodiments, the taxane in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane that is substantially free of polymeric materials (such as polymeric matrix).

In some embodiments, the composition comprises albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of albumin (such as human serum albumin) and taxane in the nanoparticle composition is about 18:1 or less, such as about 15:1 or less, for example about 10:1 or less. In some embodiments, the weight ratio of albumin (such as human serum albumin) and taxane in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of albumin and taxane in the nanoparticle portion of the composition is about any one of 1:2, 1:3, 1:4, 1:5, 1:10, 1:15, or less. In some embodiments, the weight ratio of the albumin (such as human serum albumin) and the taxane in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises human serum albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human serum albumin) in the composition generally serves as a carrier for the taxane, i.e., the albumin in the composition makes the taxane more readily suspendable in an aqueous medium or helps maintain the suspension as compared to compositions not comprising an albumin. This can avoid the use of toxic solvents (or surfactants) for solubilizing the taxane, and thereby can reduce one or more side effects of administration of the taxane into a human individual (such as a human). Thus, in some embodiments, the composition described herein is substantially free (such as free) of surfactants, such as Cremophor (including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the composition is not sufficient to cause one or more side effect(s) in a human individual when the nanoparticle composition is administered to the individual. In some embodiments, the nanoparticle composition contains less than about any one of 20%, 15%, 10%, 7.5%, 5%, 2.5%, or 1% organic solvent or surfactant.

The amount of albumin in the composition described herein will vary depending on other components in the composition. In some embodiments, the composition comprises an albumin in an amount that is sufficient to stabilize the taxane in an aqueous suspension, for example, in the form of a stable colloidal suspension (such as a stable suspension of nanoparticles). In some embodiments, the albumin is in an amount that reduces the sedimentation rate of the taxane in an aqueous medium. For particle-containing compositions, the amount of the albumin also depends on the size and density of nanoparticles of the taxane.

A taxane is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to a human individual (such as human). Stability of the suspension is generally (but not necessarily) evaluated at a storage temperature (such as room temperature (such as 20-25° C.) or refrigerated conditions (such as 4° C.)), For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize the taxane in an aqueous suspension at a certain concentration. For example, the concentration of the taxane in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of albumin, e.g., albumin, to the taxane in the nanoparticle composition is such that a sufficient amount of taxane binds to, or is transported by, the cell. While the weight ratio of albumin to taxane will have to be optimized for different albumin and taxane combinations, generally the weight ratio of albumin, e.g., albumin, to taxane (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to taxane weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human serum albumin) and the taxane in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to a human individual (such as human) without significant side effects. In some embodiments, the albumin (such as human serum albumin) is in an amount that is effective to reduce one or more side effects of administration of the taxane to a human. The term “reducing one or more side effects of administration of the taxane” refers to reduction, alleviation, elimination, or avoidance of one or more undesirable effects caused by the taxane, as well as side effects caused by delivery vehicles (such as solvents that render the taxanes suitable for injection) used to deliver the taxane. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxanes can be reduced.

In some embodiments, the nanoparticle composition comprises ABRAXANE® (Nab-paclitaxel). In some embodiments, the nanoparticle composition is ABRAXANE® (Nab-paclitaxel). ABRAXANE® is a formulation of paclitaxel stabilized by human albumin USP, which can be dispersed in directly injectable physiological solution. When dispersed in a suitable aqueous medium such as 0.9% sodium chloride injection or 5% dextrose injection, ABRAXANE® forms a stable colloidal suspension of paclitaxel. The mean particle size of the nanoparticles in the colloidal suspension is about 130 nanometers. Since HSA is freely soluble in water, ABRAXANE® can be reconstituted in a wide range of concentrations ranging from dilute (0.1 mg/ml paclitaxel) to concentrated (20 mg/ml paclitaxel), including for example about 2 mg/ml to about 8 mg/ml, about 5 mg/ml.

Methods of making nanoparticle compositions are known in the art. For example, nanoparticles containing taxanes (such as paclitaxel) and albumin (such as human serum albumin) can be prepared under conditions of high shear forces (e.g., sonication, high pressure homogenization, or the like). These methods are disclosed in, for example, U.S. Pat. Nos. 5,916,596; 6,506,405; 6,749,868; 6,537,579. 7,820,788, and also in U.S. Pat. Pub. Nos. 2007/0082838, 2006/0263434 and PCT Application WO08/137148.

Briefly, the taxane (such as paclitaxel) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1.

### Other Components in the Nanoparticle Compositions

The nanoparticles described herein can be present in a composition that include other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the nanoparticle composition (see, e.g., U.S. Pat. Nos. 5,916,596; 6,096,331; 7,820,788). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits, Medicines, and Compositions

The invention also provides kits, medicines, compositions, and unit dosage forms for use in any of the methods described herein.

Kits of the invention include one or more containers comprising taxane-containing nanoparticle compositions (or unit dosage forms and/or articles of manufacture) and/or another agent (such as the agents described herein), and in some embodiments, further comprise instructions for use in accordance with any of the methods described herein. The kit may further comprise a description of selection a human individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a) a composition comprising nanoparticles comprising a taxane (such as paclitaxel) and an albumin (such as human serum albumin), and b) instructions for administering the nanoparticle composition for treatment of solid tumor in a human individual who is no more than about 21 years old (such as no more than about 18 years old). In some embodiments, the individual has sarcoma, such as soft tissue sarcoma, for example rhabdomyosarcoma. In some embodiments, the individual has neuroblastoma.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the nanoparticle compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the taxane (such as taxane) as disclosed herein to provide effective treatment of a human individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, 10 months, 11 months, 12 months, or more. Kits may also include multiple unit doses of the taxane and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein.

### EXAMPLES

### Example 1A

### Abraxane in Pediatric Solid Tumor Xenograft Models

This example demonstrates that Abraxane® (Nab-paclitaxel) has significant anti-tumor activity against pediatric solid tumor both in vitro and in vivo.

A panel of seven neuroblastoma (NB) and three rhabdomyosarcoma (RMS) cell lines were exposed to increased concentrations of Abraxane® in vitro. Cell viability was evaluated with Alamar Blue assay. Anti-tumor effect of ABRAXANE was further assessed in vivo with xenograft models. Animal survival was also evaluated in metastatic NB models. Xenograft sections were analyzed by immunohistochemistry for cleaved caspase-3 and phospho-histone H3. In addition, plasma and intratumoral paclitaxel concentrations were measure by liquid chromatography-mass spectrometry. Ratio of intratumoral and plasma concentration was compared between Abraxane® and paclitaxel treatment groups.

Abraxane® displayed cytotoxicity against the majority of pediatric solid tumor cell lines tested in a dose-dependent manner. In vivo, Abraxane demonstrated antitumor activity in both NB (SKN-BE(2) and CHLA-20) and RMS (RH4) xenograft models. In SK-N-BE(2) metastatic model, ABRAXANE treatment significantly extended animal survival compared to control (p<0.01). It was demonstrated that Abraxane® treatment induced tumor cell cycle arrest and apoptosis in vivo. In RH4 model, increased local relapse-free intervals were observed with Abraxane® treatment (54 days) compared to paclitaxel (34 days). Local relapsed tumors following paclitaxel treatment proved to be paclitaxel-resistant remain responsive to Abraxane®. Mechanistically, elevated intratumoral and correspondingly lower plasma paclitaxel levels were observed with Abraxane® compared to paclitaxel, resulting in higher tumor/plasma paclitaxel drug ratio for Abraxane®.

Abraxane® demonstrated significant antitumor activity against pediatric solid tumors both in vitro and in vivo. Therapeutic improvement of Abraxane may be related to enhanced drug intratumoral delivery. Results of this nonclinical study support further testing of Abraxane® in clinical studies with pediatric solid tumor patient population.

### Example 1B

### Analysis of SPARC and PTEN Expression in 8 Cell Lines

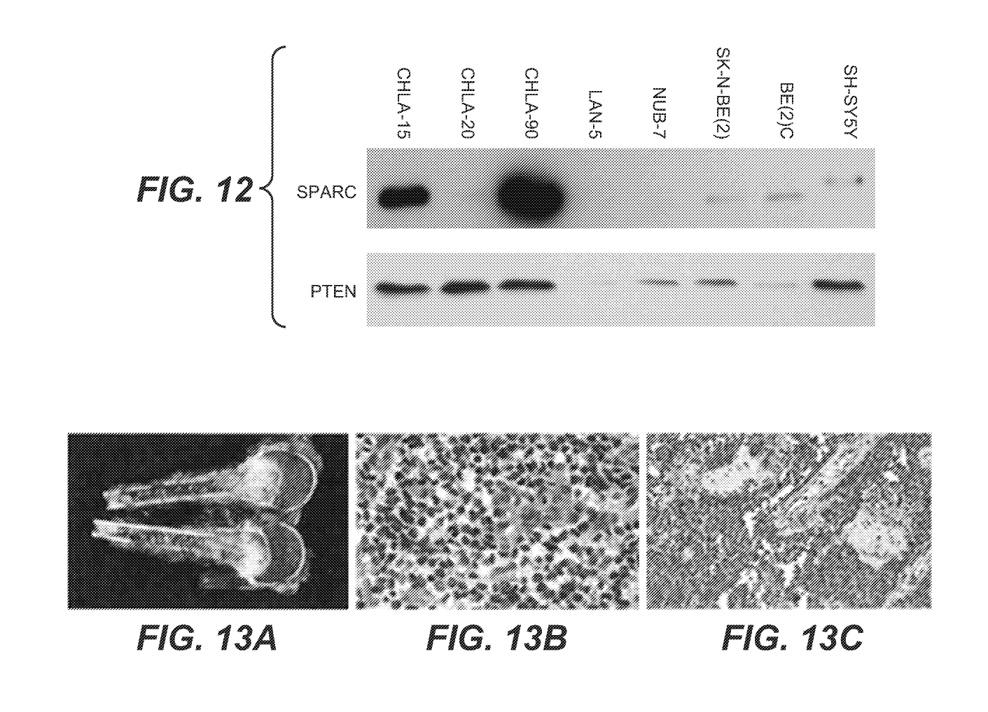

To uncover potential regulators of anti-tumor effects of Abraxane® and potential biomarkers for predicting drug response, SPARC and PTEN expression was assessed by Western blot in 8 neuroblastoma cell lines (CHLA-15, CHLA-20, CHLA-90, LAN-5, NUB-7, SK-N-BE(2), BE(2)C, and SH-SY5Y). The results are provided in FIG. 12.

### Example 1C

### Abraxane® in Preclinical Model of Pediatric Solid Tumors

Abraxane (ABI-007) was supplied as a lyophilized powder and stored at room temperature until reconstitution. Abraxane was reconstituted following the package insert with 20 ml 0.9% saline to 5 mg/ml stock solution. The dosing solutions were prepared by diluting the stock solution with 0.9% saline to the desired concentration. Taxol® (paclitaxel) was dissolved in DMSO to 25 mg/ml stock solution. The dosing solutions were prepared by diluting the stock solution with 0.9% saline to the desired concentration.

RH4, RH30 and RD rhabdomyosarcoma cells were cultured in DMEM supplemented with 10% FBS. CHLA-15, CHLA-20 and CHLA-90 neuroblastoma cells were cultured in Iscove's modified Dulbecco's medium supplemented with 3 mM 1-glutamine, insulin, and transferrin 5 μg/ml each and 5 ng/ml selenous acid and 20% fetal bovine serum (FBS, complete medium). LAN-5, SK-N-BE(2), BE(2)C, and SH-SY5Y neuroblastoma cells were cultured in AMEM with 10% FBS. KHOS osteosarcoma cells were cultured in Eagle's Minimum Essential Medium supplemented with 10% FBS.

Cells were seeded into 24-well tissue culture plates at a density of 200,000 cells/well in culture medium and incubated for 24 hours at 37° C. before starting drug treatment. Cells were exposed to increasing concentrations of Abraxane® (10⁻³-10³ ng/ml) for 72 hours. The viability of proliferating cells in the control and treated media were measured with an Alamar Blue assay according to manufacturer's protocol. Briefly, Alamar Blue was diluted 1 to 10 in the cell culture media, and the fluorescent color change was monitored after 3 hours. Colorimetrical evaluation of cell proliferation was performed using a SPECTRAmax Gemini spectrophotometer with 540 nm as excitation wavelength and 590 nm as emission wavelength and values expressed as Relative Fluorescence Units (RFU). Cell viability was measured in triplicate and calculated relative to control non-treated cells.

Annexin V was used to detect apoptosis with the Annexin V-FITC Early Apoptosis Detection Kit. Cells were cultured (2×10⁵ cells) on coverslips overnight prior to the treatment with Abraxane® for 48 hours. For apoptosis staining with annexin V-FITC, after incubated with Annexin V-FITC according to manufacturer's protocol, the cells were washed and fixed in 2% formaldehyde before visualization under a fluorescence microscope using a dual filter set for FITC-Annexin V and DAPI (nuclei staining).

The antitumor activity of Abraxane®/Taxol® was investigated in vivo against subcutaneous rhabdomyosarcoma (RH4 and RH30) and neuroblastoma (SK-N-BE(2) and CHLA-20) using NOD/SCID tumor xenografts. Briefly, tumor cells were washed three times with HBSS before injection. Mice were given a subcutaneous injection of 1×10⁶ tumor cells. Tumor growth was measured weekly in two dimensions using a digital caliper, and tumor volume was calculated as width²×length×0.5. Once the tumor diameter reached 0.5 cm, mice were randomized into treatment groups with 10 animals in each group. Abraxane® was administered either at low-dose metronomic administration (three different doses of 2, 5, or 10 mg/kg i.v. daily) or cytotoxic dose (50 mg/kg i.v. weekly). Taxol® was administered i.v. at 20 or 30 mg/kg weekly. Control mice received saline. Tumor volume, mouse body weight and signs of animal distress were evaluated twice or three times a week for any potential drug toxicity. Animals were sacrificed once the tumor size reached 1.5 cm³.

The anti-metastatic activity of Abraxane was further investigated in SK-N-BE(2) neuroblastoma metastatic models. Tumor cells were injected intravenously into the lateral tail vein (26-gauge needle, 1×106 cells in 100 total volume). Mice were randomized into 2 groups (control and Abraxane 50 mg/kg iv weekly) with 10 mice in each group and treatments started 14 days after inoculation until the event of endpoint. The event of endpoint was defined according to our animal committee guidelines as mice in severe clinical condition, such as loss of 20% of body weight, body temperature lower than 32° C., or signs of stress. The survival time of control and Abraxane® treatment groups was compared and statistically analyzed.

In order to assess the effect of Abraxane® on inducing cell cycle arrest and apoptosis in vivo, SK-N-BE(2) subcutaneous xenografts treated with Abraxane® or DMSO-Taxol® were harvested at the end of study and analyzed by immunohistochemistry (IHC) for the apoptotic marker (cleaved caspase-3) and mitotic marker (phospho-histone H3) following instruction by manufacturers. Similarly, RH4 xenografts were harvested and analyzed by IHC for phosphor-histone H3.

Plasma and intratumor drug concentration was studied after single or repeated drug administration. In RH4 xenograft model, blood/tumor samples were collected 24 hours after the first dosage of Abraxane® (50 mg/kg) or Taxol® (30 mg/kg). In SK-N-BE(2) xenograft model, Taxol® (20 mg/kg) and Abraxane® (50 mg/kg) were administered on day 1, 8 and 15. Low-dose metronomic Abraxane® (10 mg/kg) was administered daily from day 1 to day 15. 24 hours after the last dosage of Abraxane®/Taxol®, blood and tumor samples were collected and analyzed for Taxol® concentration by LC/MS. Ratio of intratumoral vs plasma concentration was calculated and compared between Abraxane and DMSO-based Taxol® treatment groups.

Data from different experiments were presented as mean±SD. For statistical analysis, Student's t test for independent means was used. A P value of <0.05 was considered significant. To compare the effects of different treatments on tumor growth in vivo, one-way ANOVA with Dunnett multiple comparison test was used. Survival curve comparisons were performed using Graphpad Prism software for Kaplan-Meier Survival Analysis.

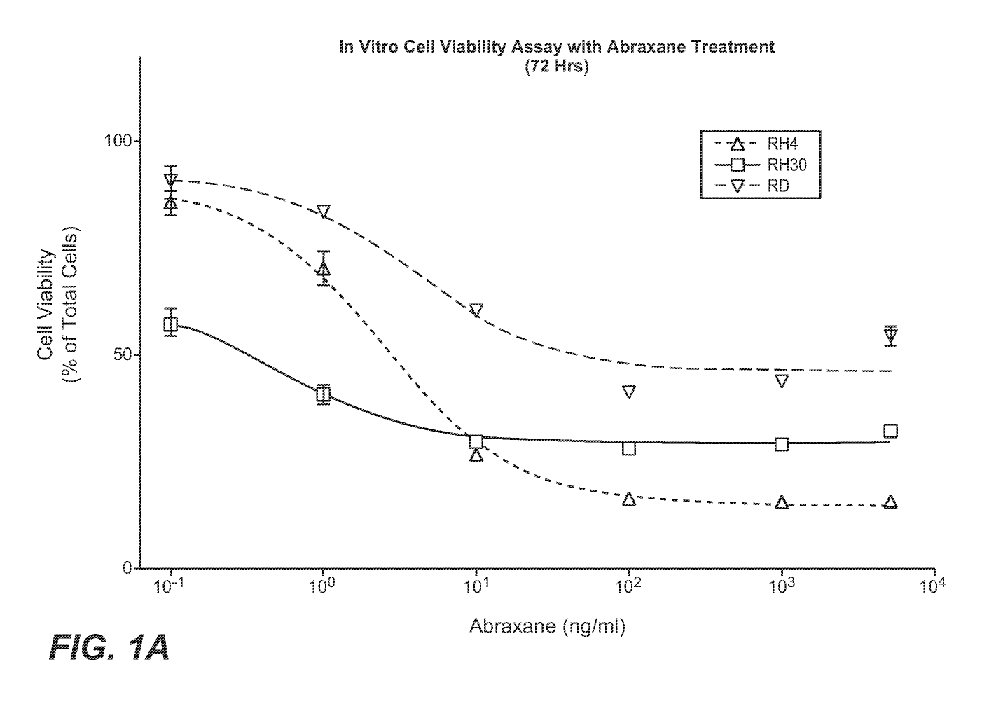

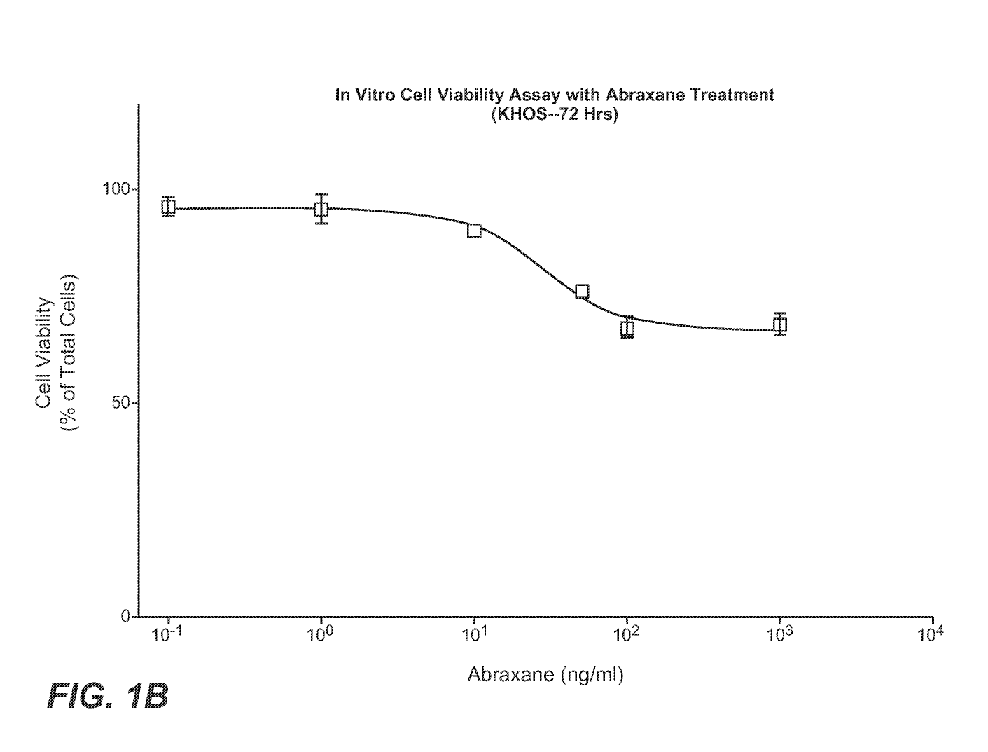

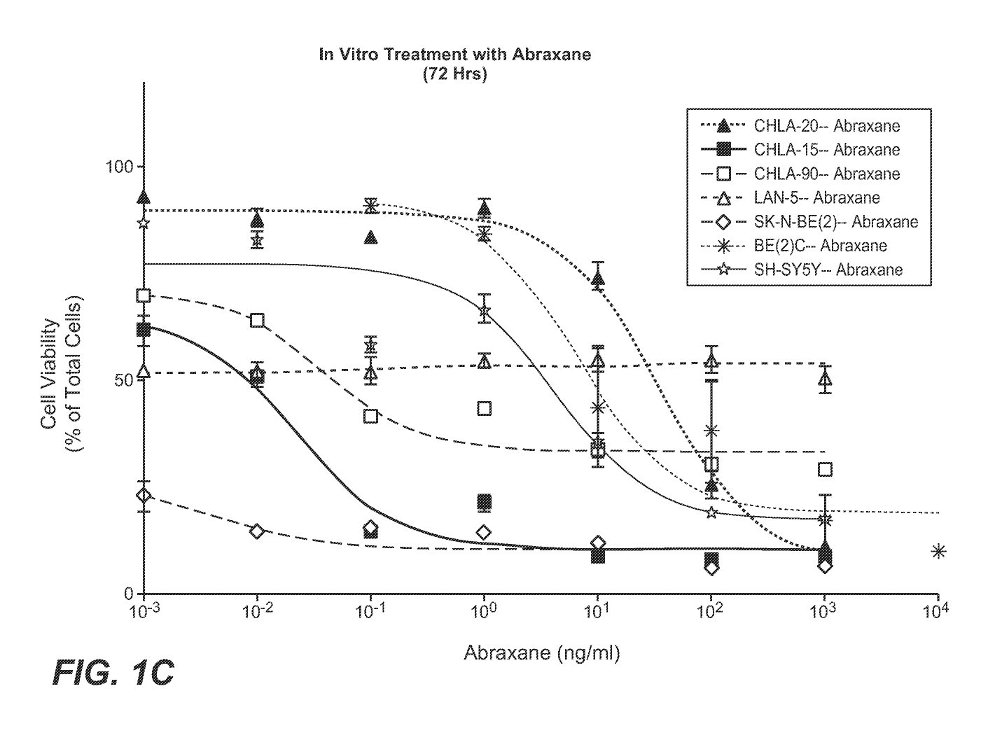

To determine the efficacy of Abraxane against a wide panel of pediatric cancer cells, 3 rhabdomyosarcoma (RH4, RH30 and RD), 7 neuroblastoma cell lines (CHLA-20, CHLA-15, CHLA-90, LAN-5, SK-N-BE(2), BE(2)C, and SH-SY5Y), and 1 osteosarcoma cell line (KHOS), were tested for viability with Alamar Blue assays after exposing cells to increasing concentrations of Abraxane® in vitro for 72 hours. As shown in FIG. 1A, all three rhabdomyosarcoma cell lines were responsive to Abraxane® treatment in a dose-dependent manner. IC50 values were calculated and ranged from 0.48 to 4.0 ng/ml. Limited response was observed with osteosarcoma cell line KHOS (FIG. 1B).

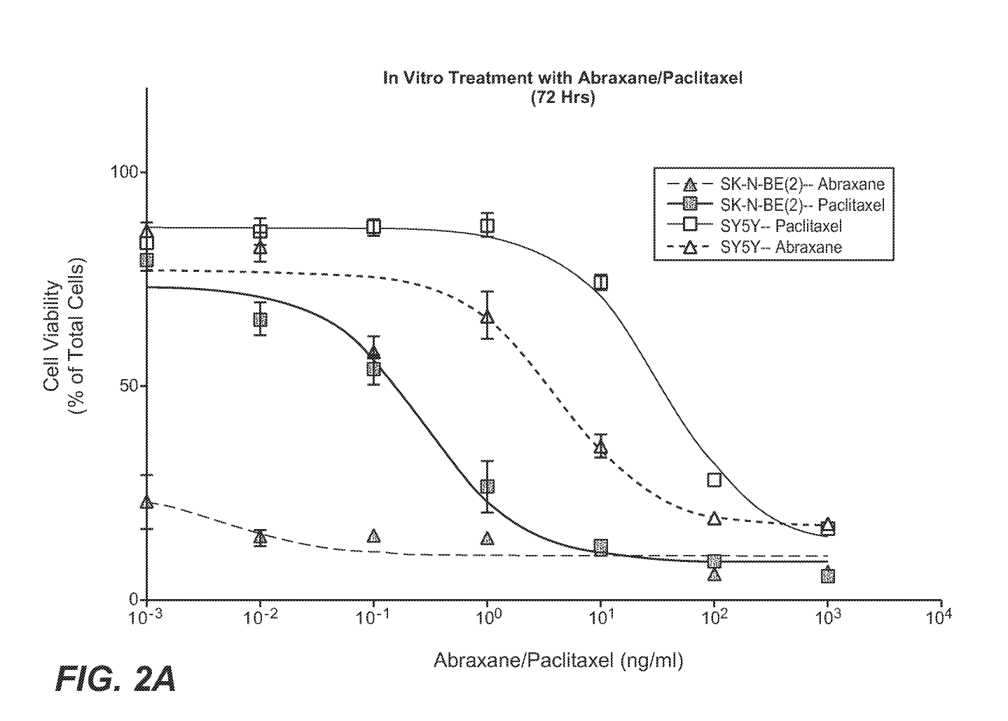

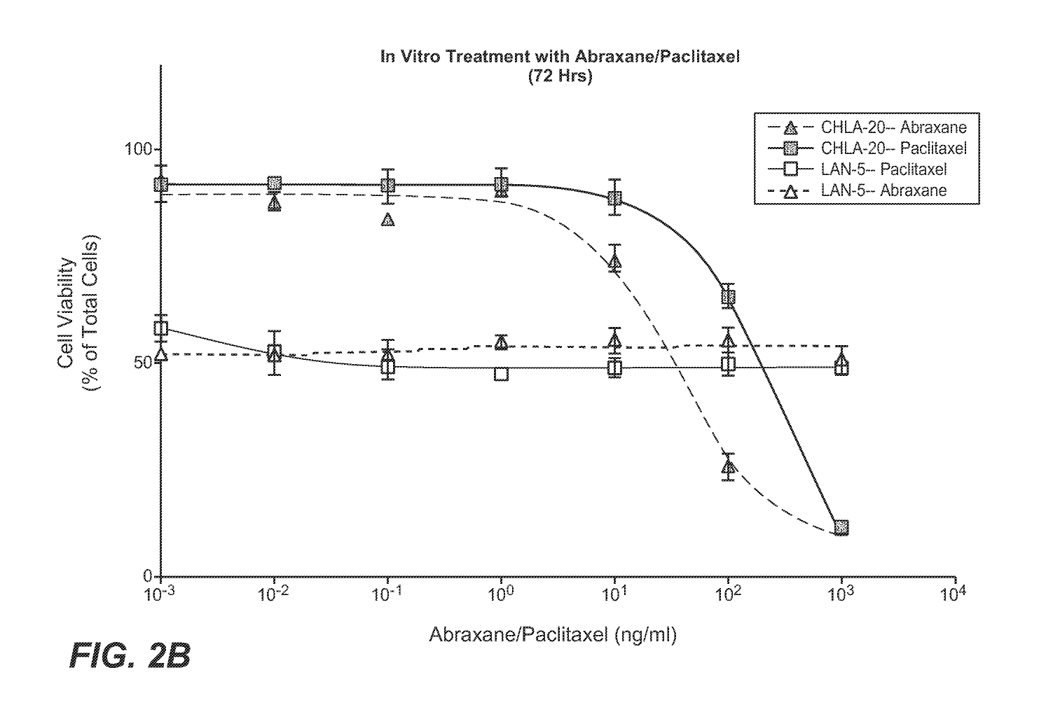

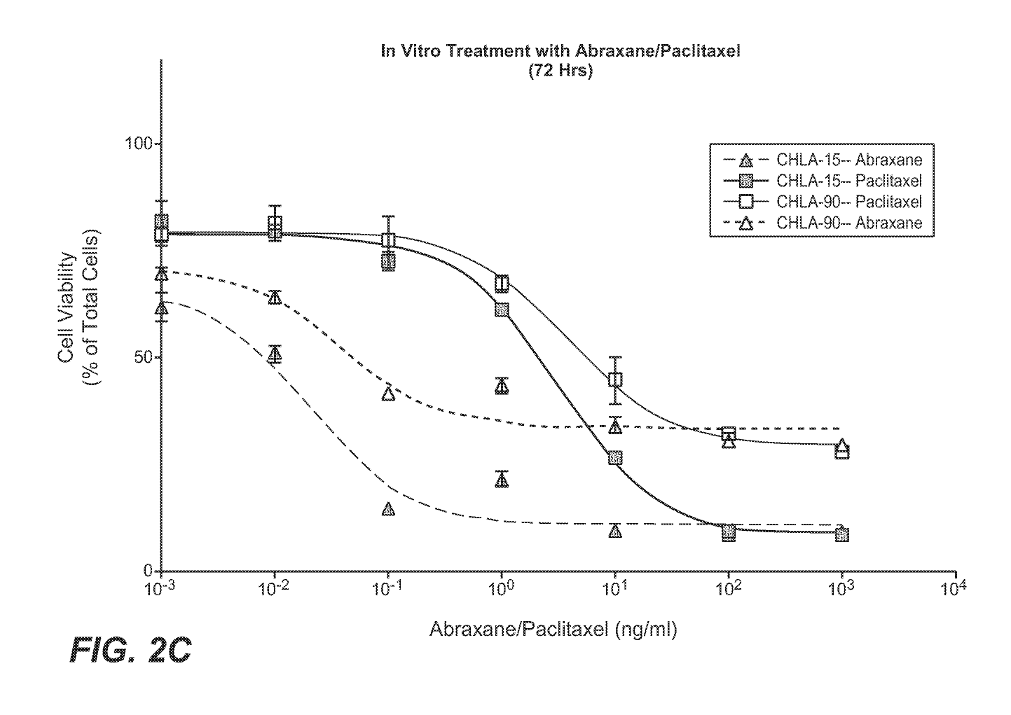

For the seven neuroblastoma cell lines, Abraxane® exhibited dose-dependent cytotoxicity in vitro, as measured by cell viability (FIG. 1C). Different cell lines displayed variable sensitivity for Abraxane®. Among all these cell lines, CHLA-20 has the highest EC50 (36 nM), while LAN-5 and SK-N-BE(2) have the lowest EC50. Furthermore, when neuroblastoma cell lines were treated for 72 hours in vitro, all the tested cell lines showed more sensitivity to Abraxane® than to Taxol® dissolved in the solvent DMSO (FIGS. 2A-C), suggesting that Taxol® in albumin-bound formulation in solution more readily available for tumor cell uptake.

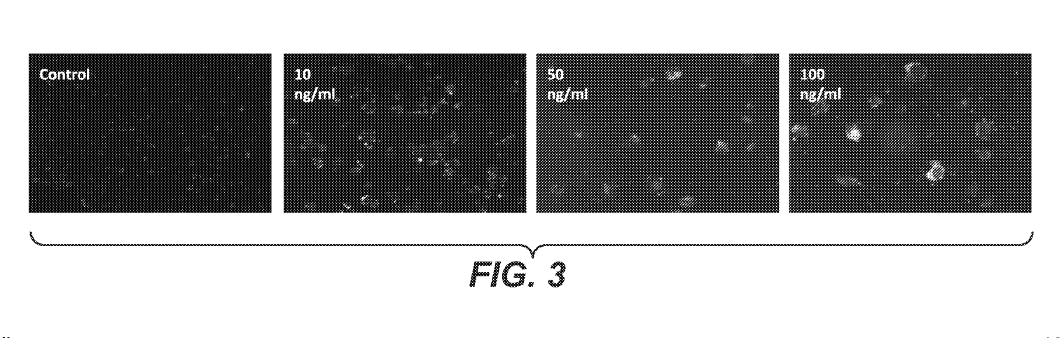

We further assessed cell apoptosis after in vitro drug treatment. Rhabdomyosarcoma RH4 cells were incubated with increased concentration of Abraxane® (i.e., 10, 50 or 100 ng/ml) for 48 hours and analyzed for apoptosis with annexin V-FITC. Annexin V-FITC conjugated protein binds to cell surfaces expressing phosphatidylserine, an early apoptosis marker. Increased apoptotic RH4 cells as shown by annexin V-FITC positive staining were observed following Abraxane® treatment (FIG. 3). With the higher concentration of Abraxane® (50 or 100 ng/ml), most cells detached from the coverslips, but almost all of the remaining cells showed annexin V-FITC positive staining.

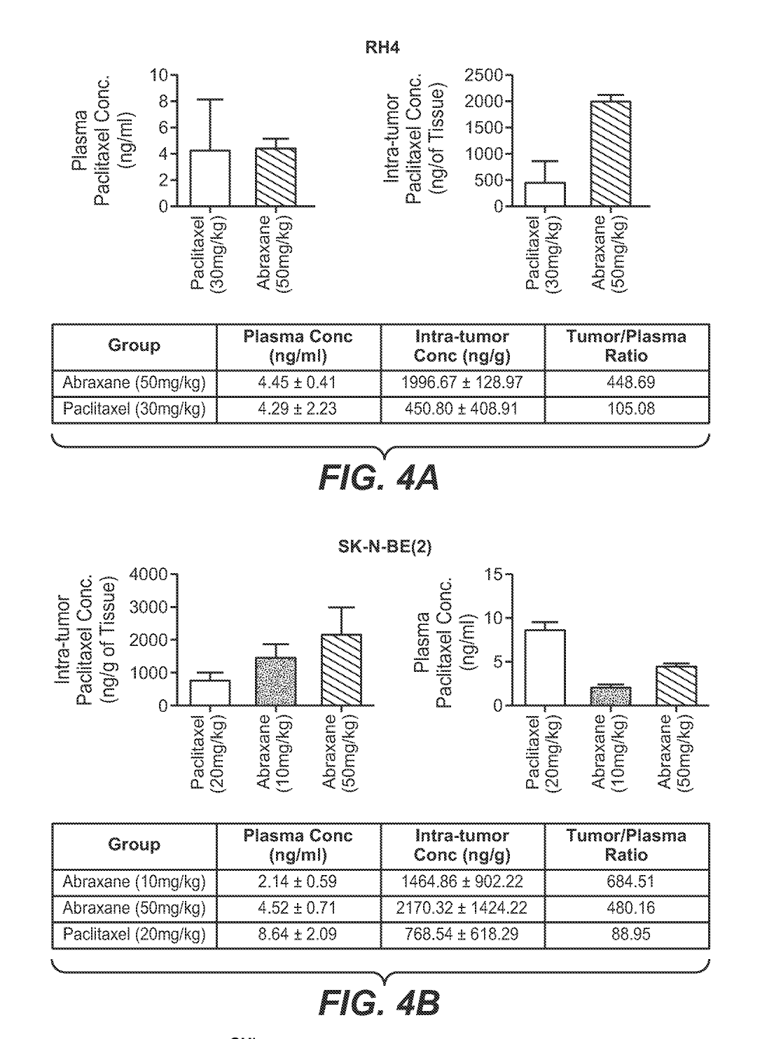

Plasma and intratumor drug concentration was measured after single or repeated drug administration. Mice bearing human rhabdomyosarcoma (RH4) and neuroblastoma (SK-N-BE(2)) xenografts were intravenously administered different dosages of Taxol® (20 mg/kg or 30 mg/kg weekly) or Abraxane® (10 mg/kg/day for 5 consecutive days or 50 mg/kg weekly). Twenty-four hours after the last dose, blood and tumor samples were collected and analyzed for Taxol® concentration by LC/MS. In both tumor models, Abraxane® treatment displayed lower plasma Taxol® concentrations compared to DMSO-Taxol®, whereas the intratumor Taxol® concentrations were higher with Abraxane® groups (FIGS. 4A and 4B), As a consequence, Abraxane® had a higher tumor/plasma Taxol® ratio compared to DMSO-Taxol® 24 hours after drug administration.

The in vivo antitumor activity of Abraxane® was evaluated in multiple pediatric tumor xenografts. In rhabdomyosarcoma models, mice bearing RH4 and RD xenografts were treated intravenously with Abraxane® (50 mg/kg) and Taxol® (30 mg/kg). The 50 mg/kg weekly dosing corresponds to 150 mg/m² weekly dosing in humans, which is the highest dose for weekly Abraxane® treatment in adults. 30 mg/kg of Taxol® corresponds to the highest dosage in adult patients too.

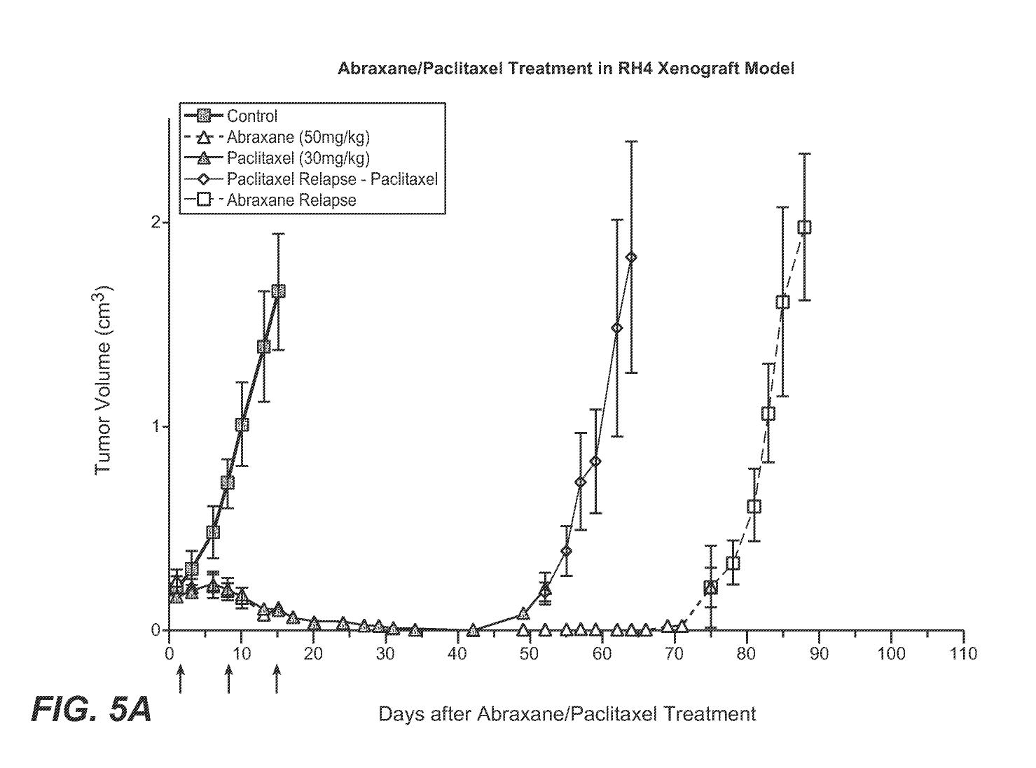

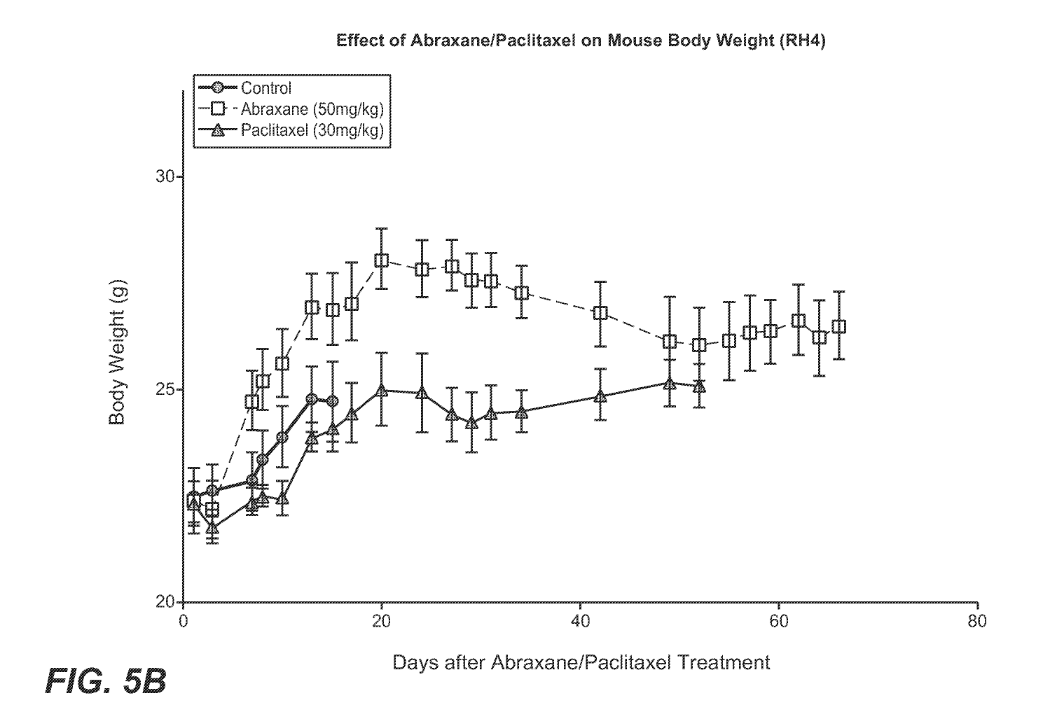

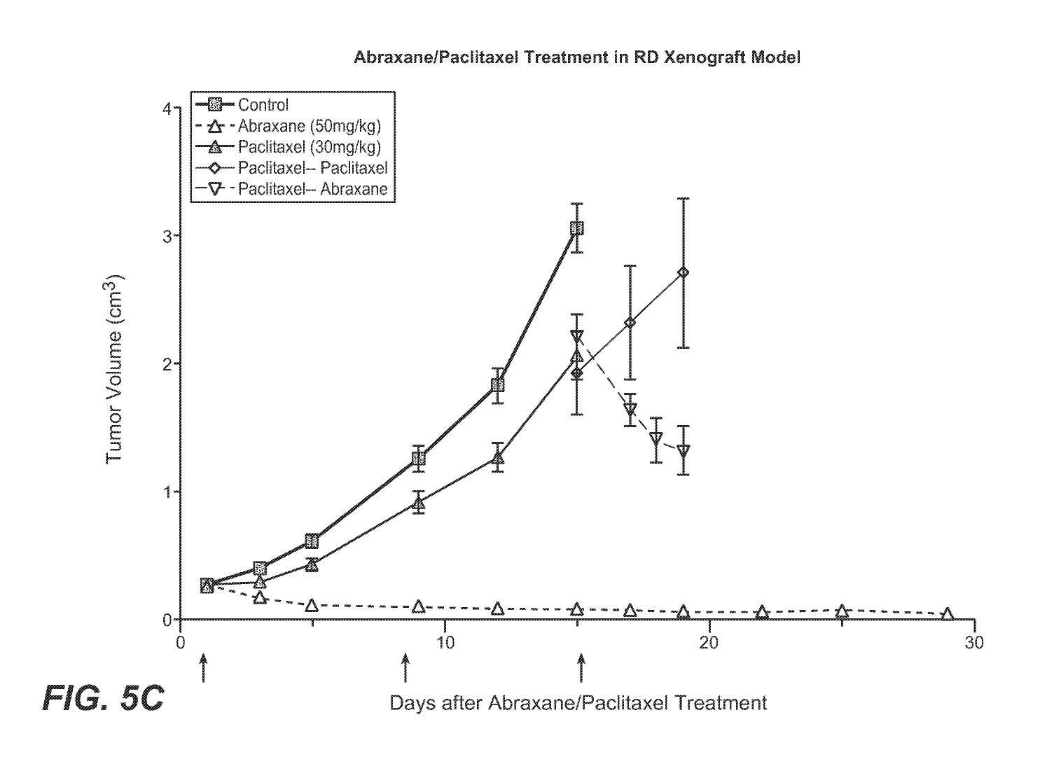

Both Abraxane® and DMSO-Taxol® treatments significantly inhibited RH4 tumor growth, with tumor regression observed after the 2nd dosage on day 8 (FIG. 5A). However, animals treated with Taxol® showed lower body weight compared to Abraxane and control animals (FIG. 5B), and 1 out of 7 mice in Taxol® group died on Day 10, demonstrating that Taxol®, even at a lower dose, had higher toxicity compared with Abraxane®. In the RH4 model, increased local relapse-free intervals were observed with Abraxane® treatment (37.7±3.2 days) comparing to Taxol® (13.6±2.07 days).

In the RD xenograft model, Taxol® (30 mg/kg, weekly) or Abraxane® (50 mg/kg, weekly) was administered on days 1 and 8. Tumor regression was observed with Abraxane® treatment, Compared to control animals, Taxol® treatment was able to slow the growth of RD tumors, but those tumors grew progressively with no signs of tumor regression. On day 15, when the Taxol® drug treatment was replaced with Abraxane® (50 mg/kg, weekly), those tumors regressed rapidly after the first dosage of Abraxane (FIG. 5C).

Tumor growth was assessed in RD xenograft model with paclitaxel/Abraxane treatment. In Abraxane treatment group, tumor-bearing mice received Abraxane (50 mg/kg, weekly) treatment. In paclitaxel treatment group, since tumor sizes are reaching the endpoint after 2-week paclitaxel treatment in paclitaxel group, those mice were randomized into two groups with 5 animals in each group on day 15: one group of animals continued receiving 30 mg/kg of paclitaxel and the other group received 50 mg/kg of Abraxane instead. In RD xenograft model, both Taxol® and Abraxane® treatment significantly inhibited tumor growth, but tumor shrinkage was only observed in Abraxane® treated tumors (FIG. 5C).

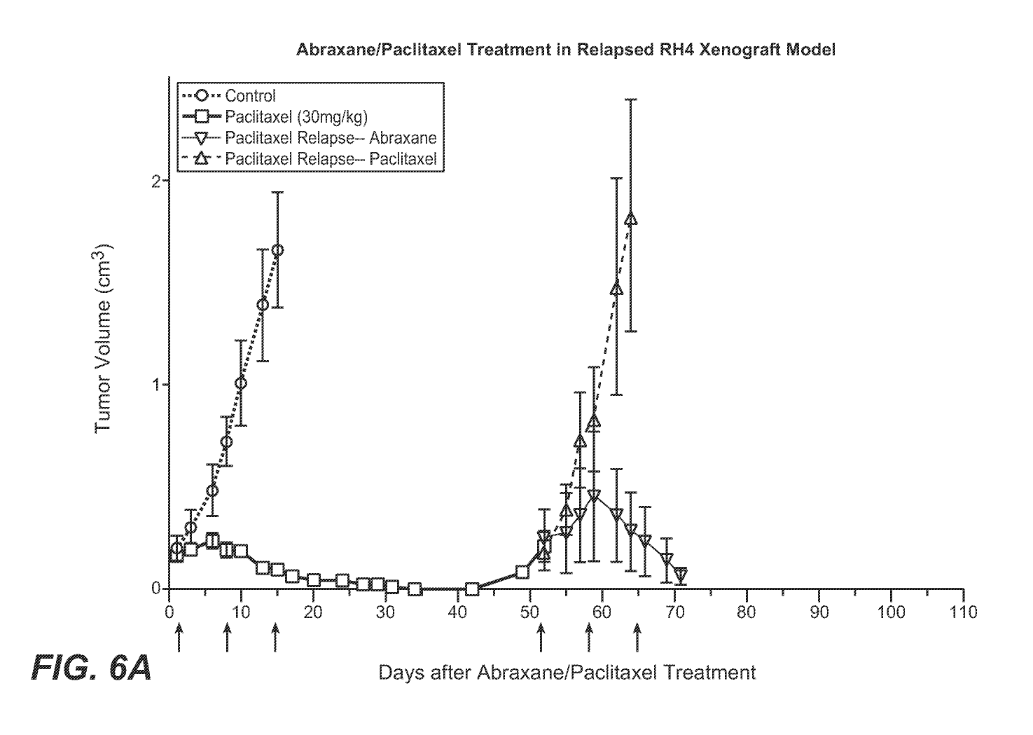

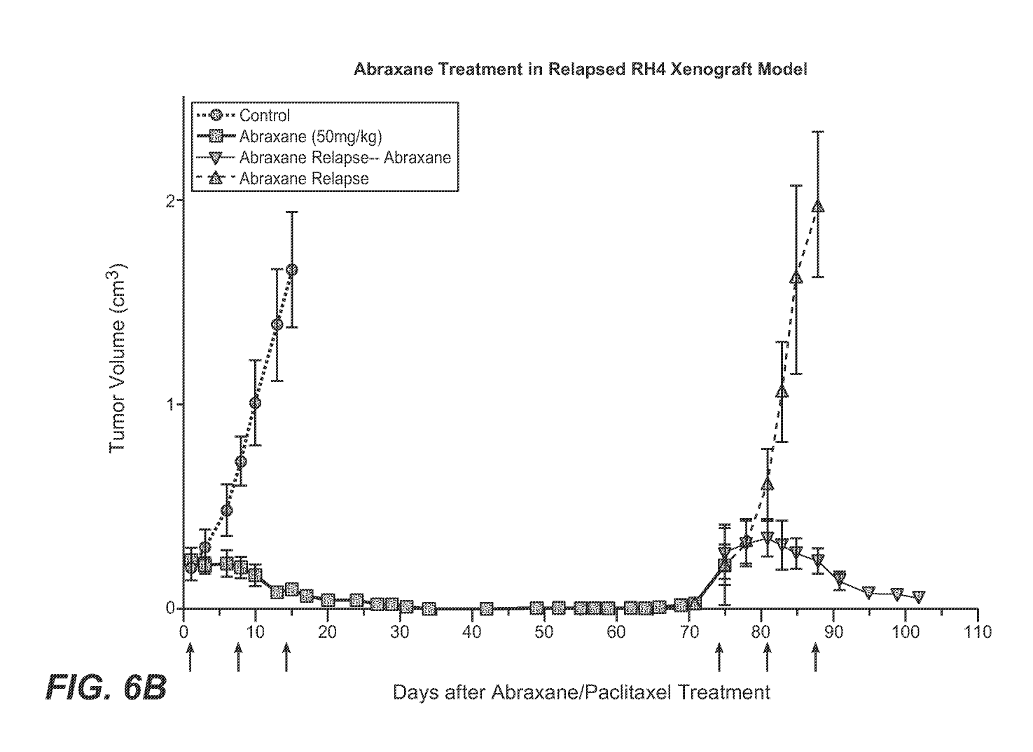

In RH4 xenografts when tumors reached above 0.5 cm in diameter, mice were randomized into three groups (controlled, Abraxane treatment, and paclitaxel treatment) with 7 animals in each group. Taxol® (30 mg/kg) or Abraxane® (50 mg/kg) was administered on days 1, 8 and 15. Tumor volume was measured and calculated as width²×length×0.5. Complete regression was observed in Taxol® treated mice after day 31 (FIG. 6A). However, all Taxol® treated animals demonstrated tumor relapse after 11-15 days. On day 52, when Taxol® relapsed tumors reached 0.5 cm in diameter, animals were randomized into two treatment groups: Abraxane® and Taxol®. Drugs were given on day 52, 59 and 66 with the same schedule and dosage as above. Tumor growth was monitored. As shown in FIG. 6A, relapsed RH4 xenografts were drug resistant against Taxol®, but remained sensitive to Abraxane® treatment. Tumor regression was observed in all relapsed tumors which were treated again with Abraxane®.

Complete regression was observed in Abraxane® treated mice after day 29 (FIG. 6B). Six out of seven animals developed relapsed tumors after 37-42 days. On day 75, when Abraxane® relapsed tumors reached 0.5 cm in diameter, animals were randomized into two groups: Abraxane® treatment and saline control. Abraxane® or saline was given on day 75, 82 and 87 with the same schedule and dosage as above. As seen in FIG. 6B, when those relapsed RH4 tumors were treated with Abraxane® (50 mg/kg, weekly) again, relapsed tumors from Abraxane® remained responsive to Abraxane® as demonstrated in FIG. 6B.

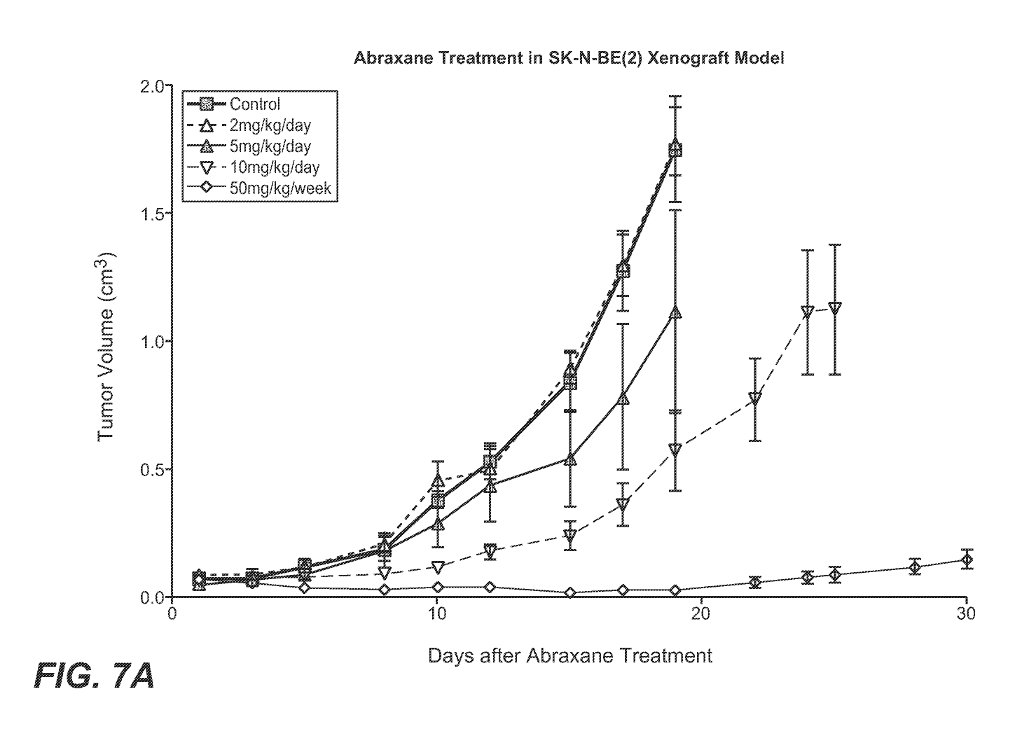

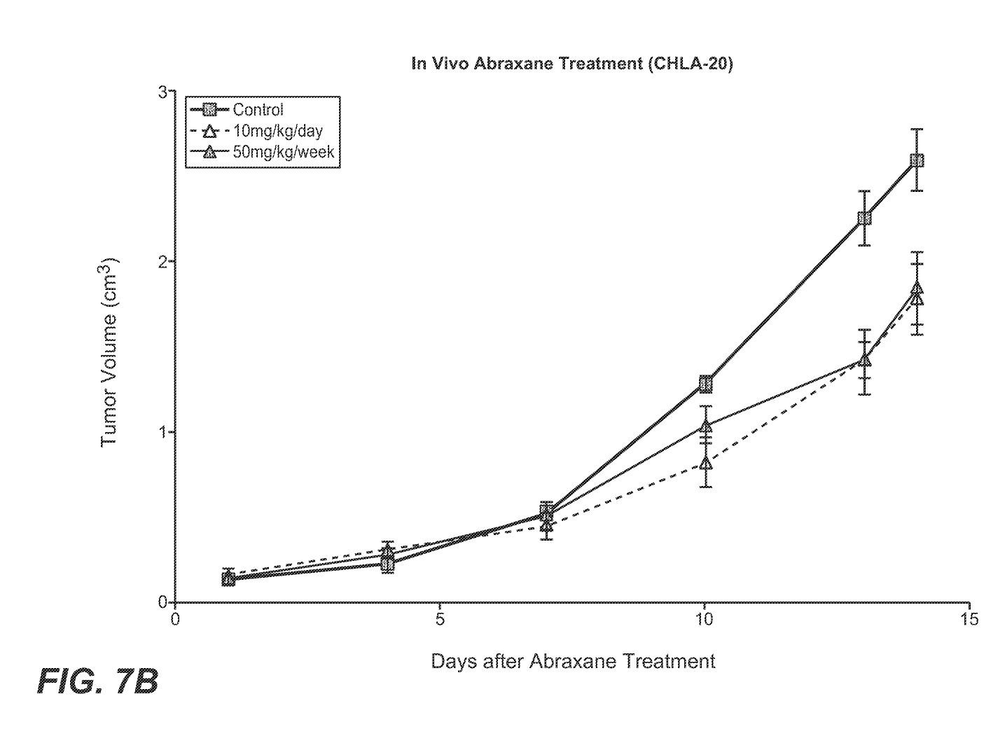

Different schedules and doses of Abraxane® (i.e., low-dose metronomic (LDM) and standard maximum tolerated dose (MTD) schedule) were compared in neuroblastoma xenograft models. Subcutaneous mouse xenograft tumors (SK-N-BE(2) and CHLA-20) were treated with either vehicle alone, Abraxane® at 2, 5, and 10 mg/kg daily or 50 mg/kg weekly. Control mice received saline. Increasing doses of Abraxane® at 2, 5, 10 mg/kg iv daily clearly demonstrated greater tumor growth inhibition with SK-N-BE(2) in a dose-dependent manner (FIG. 7A). The 2 mg/kg/day dosage showed no significant effect on tumor growth, while the 5 and 10 mg/kg daily doses significantly inhibited tumor growth. The strongest anti-tumor activity was observed with Abraxane® at 50 mg/kg iv weekly. Tumor growth was also evaluated in CHLA-20 xenograft model. Tumor bearing mice were treated with either standard maximum tolerated dose of Abraxane (MTD; 50 mg/kg, weekly) or low-dose metronomic Abraxane (LDM; 10 mg/kg, daily). In the CHLA-20 xenograft model, Abraxane® at 50 mg/kg iv weekly demonstrated similar antitumor activity compared with LDM therapy at 10 mg/kg daily (FIG. 7B).

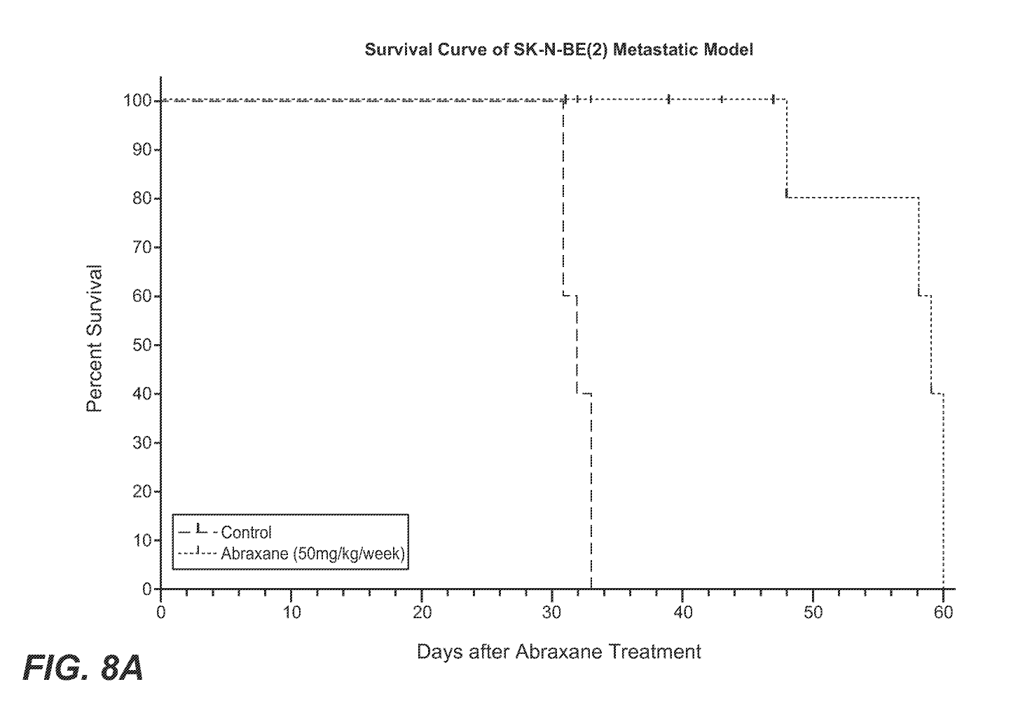

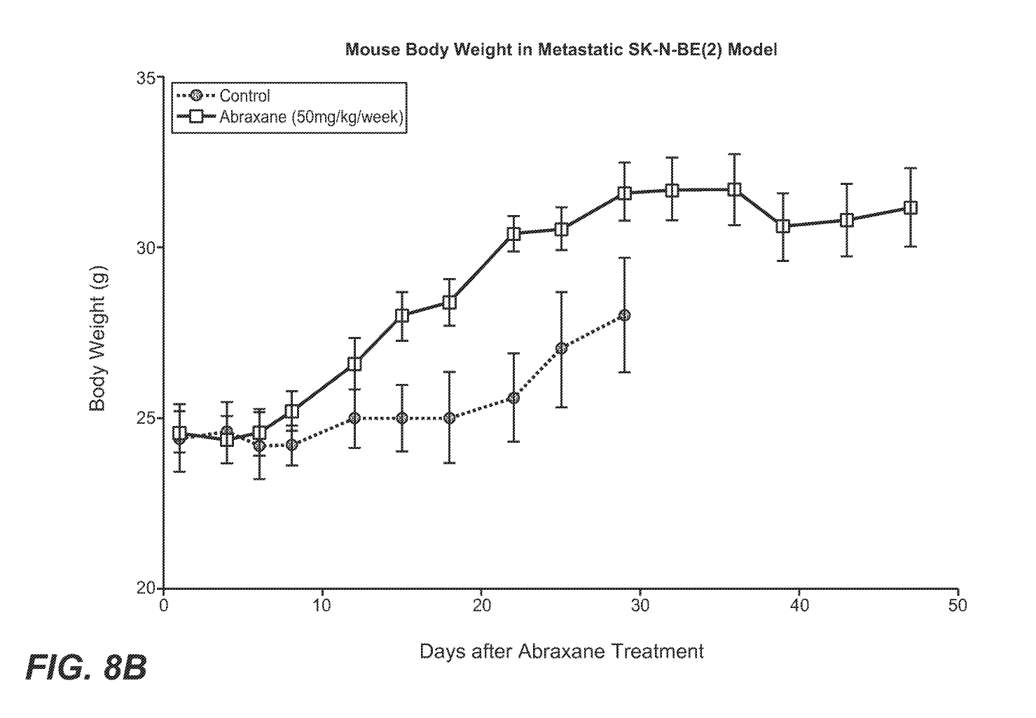

The animal survival from Abraxane® treatment was further investigated in SK-N-BE(2) metastatic models. Tumor-bearing mice were treated with control vehicle or Abraxane® (50 mg/kg iv weekly) with all treatments starting 14 days after tumor cell inoculation. As shown in FIG. 8A, Abraxane® treatment significantly prolonged animal survival compared with the control group (59 days' median survival for Abraxane group vs 32 days for control group; P<0.01). Abraxane® treatment significantly increased body weight in these mice (FIG. 8B) compared to control.

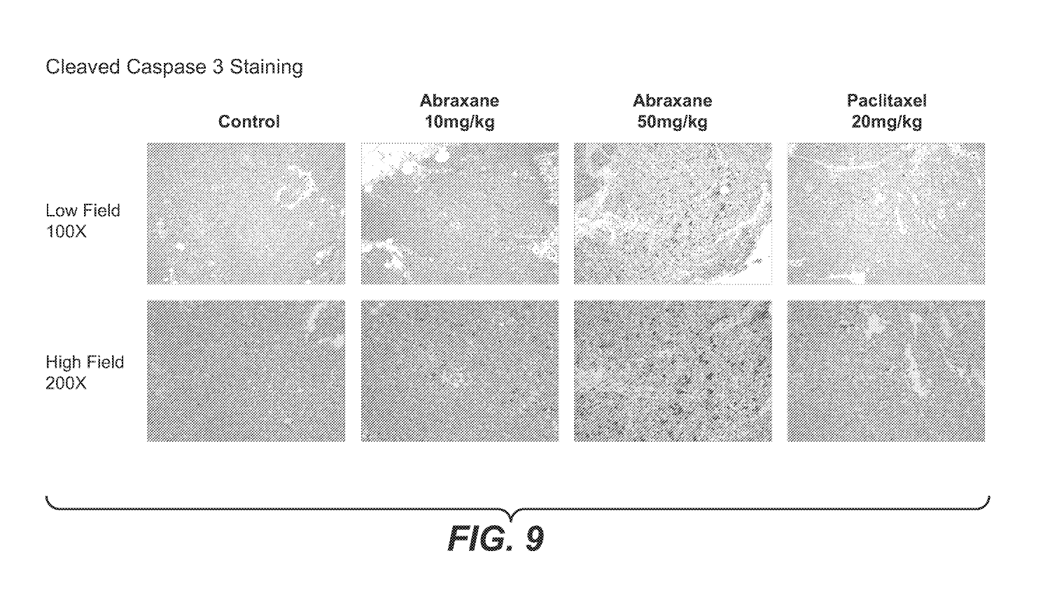

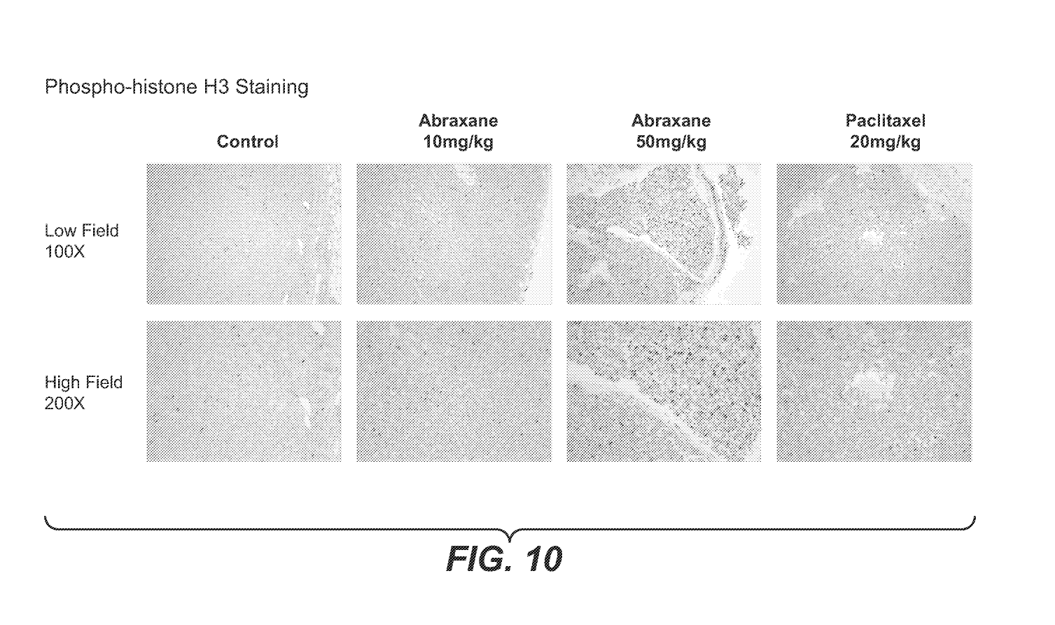

To determine whether the anti-tumor activity of Abraxane® was the result of tumor cells apoptosis and cell cycle arrest, SK-N-BE(2) xenografts treated with different dosages of Abraxane® or Taxol® were harvested at the end of study and analyzed by immunohistochemistry (IHC) for the apoptotic marker (cleaved caspase-3) and mitotic marker (phospho-histone H3). Corresponding with results of tumor growth inhibition, Abraxane® treatment significantly increased apoptotic cell population in a dose-dependent manner compared to control tumors, whereas Taxol® at 20 mg/kg/weekly only slightly increased apoptosis in tumors (FIG. 9). Similarly, Abraxane® treatment also increased phospho-histone H3 positive cells in a dose-dependent manner (FIG. 10). Taxol® at 20 mg/kg/weekly only slightly increased phospho-histone H3 positive cells in tumors.

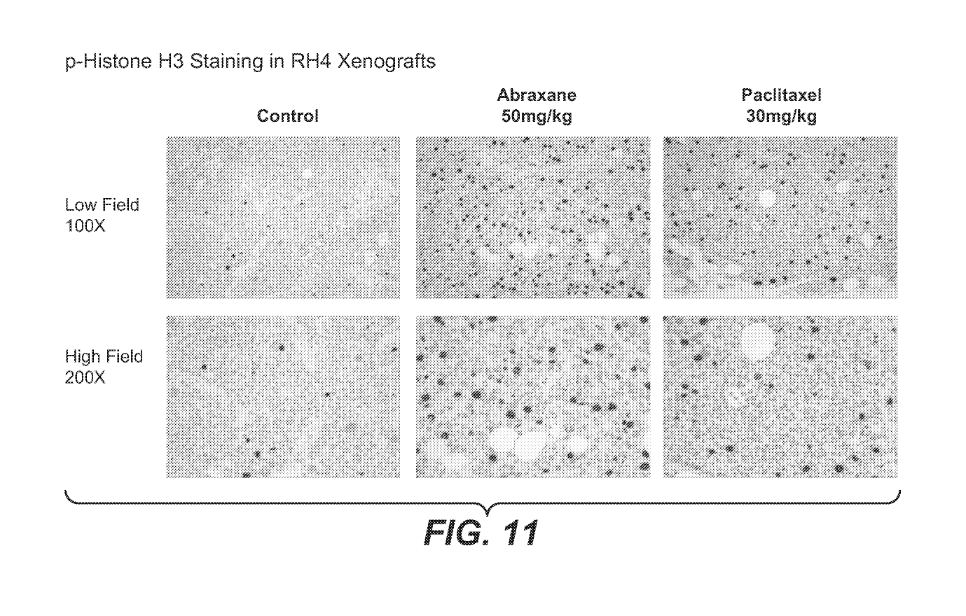

In a separate experiment, RH4 xenografts were harvested 48 hours after administering Abraxane® (50 mg/kg, iv) or Taxol® (30 mg/kg, iv), and tumor sections were stained for phospho-histone H3 by IHC. Significant increased population of phospho-histone H3 positive cells were observed after Abraxane® and Taxol® treatment (FIG. 11). Abraxane® demonstrated significant antitumor activity against pediatric solid tumors both in vitro and in vivo. Therapeutic improvement of Abraxane® may be related to enhanced drug intratumor delivery. Results of this pre-clinical study support further testing of Abraxane® in pediatric solid tumor patient population.

### Example 2

### A Phase I/II Study of Abraxane® in Treating Pediatric Cancers

This Example reports a Phase I dose-finding study to evaluate the maximum tolerated dose (MTD) and dose limiting toxicities (DLTs) of Abraxane® in patients with childhood solid tumor malignancies (e.g., rhabdomyosarcoma (RMS), neuroblastoma (NB), or other tumor types, such as non-RMS soft tissue sarcomas and melanomas). Following baseline evaluations, patients (12-24 patients whose ages range between 6 months and 21 years) enter into the treatment period. The patients have failed first- or second-line treatment or have evidence of refractory disease, and exhibit taxane-refractory solid tumors (except brain tumors). Abraxane® is administered by intravenous infusion for 30 minutes weekly for 3 weeks followed by 1 week of rest (28-day cycle), with a starting dose of 120 mg/m². The starting dose of Abraxane® was chosen based upon nonclinical toxicology data.

The first cycle is considered the treatment interval for determination of DLTs and the MTD. The MTD for Abraxane® is determined using a standard 3+3 design, where 3 patients are enrolled at each dose level. If no DLT is observed, 3 additional patients are enrolled at the next dose level. If 1 DLT is observed, the dose level is expanded to 6 patients. If 2 DLTs are observed at a given dose level, the MTD is considered to be exceeded. Of the 6-patient expanded cohort, if ≦1 out of 6 patients experiences a DLT, this is defined as the MTD. All patients at a given dose level complete 1 cycle of therapy before patients are enrolled at the next dose level.

A DLT is defined (using the National Cancer institute Common Terminology Criteria of Adverse Events [NCI CTCAE] v3.0 as any grade 3/4 nonhematologic toxicity, grade 3/4 nausea or vomiting that occurs despite treatment, grade 4 thrombocytopenia of any duration and grade 4 uncomplicated neutropenia (i.e., without fever or infection) lasting >7 days, grade 4 febrile neutropenia that requires hospitalization, and any grade 3 hematologic toxicity that requires treatment delay beyond 3 weeks.

Throughout the study, patients are routinely assessed for toxicities, response assessments, and possible need for a dose modification. Patients continue on treatment until they experience progressive disease (PD) or unacceptable toxicity, withdraw consent, or their physician feels it is no longer in their best interest to continue on treatment. Discontinued patients complete the end of study evaluation and enter into a 30-day follow-up period.

### Example 3

### Preclinical Evaluation of Nanoparticle Albumin-Bound Paclitaxel for Treatment of Pediatric Bone Sarcoma

SPARC was expressed in the majority of 25 Ewing sarcoma primary tumors, including 10 (40%) with extensive expression (scores of 3, FIG. 13), and another 3 (12%) with more limited expression. Extensive SPARC expression was seen in all 7 samples taken from patients with recurrent Ewing sarcoma.

Testing with anti-Osteonectin/SPARC antibody was performed using a 1:100 dilution. Ewing sarcoma tumor tissue was analyzed using 4 μm formalin-fixed, paraffin embedded tissue sections and a Ventana Discovery automated immunostainer, with standard immunoperoxidase techniques employed. Protein expression in tumor tissue was scored in a semiquantitative fashion incorporating both intensity and extent of staining, defined on a scale of 0-4 (0=no expression, 1=<10% of tumor cells stained, 2=10-50%, 3=50-80%, and 4=>80%). Staining intensity was graded as follows: 0=no staining; 1=weak, light yellow staining; 2=moderate, yellow-brown staining; and 3=brown, strong staining. An immunoreactivity score was calculated by dividing the sum of the individual staining intensities observed in the tissue cylinders of a single case by the number of cylinders available from each case, as described in Remmele et al. “Recommendation for uniform definition of an immunoreactive score for immunohistochemical estrogen receptor detection in breast cancer tissue.” Pathologie 1987; 8: 138-140.

Mice bearing 143.98.2 osteosarcoma cells were treated with gemcitabine, nab-paclitaxel (i.e., Abraxane®), or the two drug combination. Briefly, 5×10⁶143.98.2 osteosarcoma cells or A673 Ewing's sarcoma cells were suspended in 100 μl PBS and implanted subcutaneously with 33% Matrigel in 5-6 week-old female athymic nu/nu mice. Tumor volume was calculated using the formula L·W2·(π/6), where L=the longest tumor diameter and W=the widest tumor diameter perpendicular to L. When tumors reached 200-300 mm³, animals were treated with either saline control, nab-paclitaxel 30 mg/kg intravenously on days 1-5 for a single course, gemcitabine 100 mg/kg intraperitoneally twice weekly until death, or the combination of nab-paclitaxel and gemcitabine. Mice were sacrificed once tumors reached 10% body weight (˜2500 mm³).

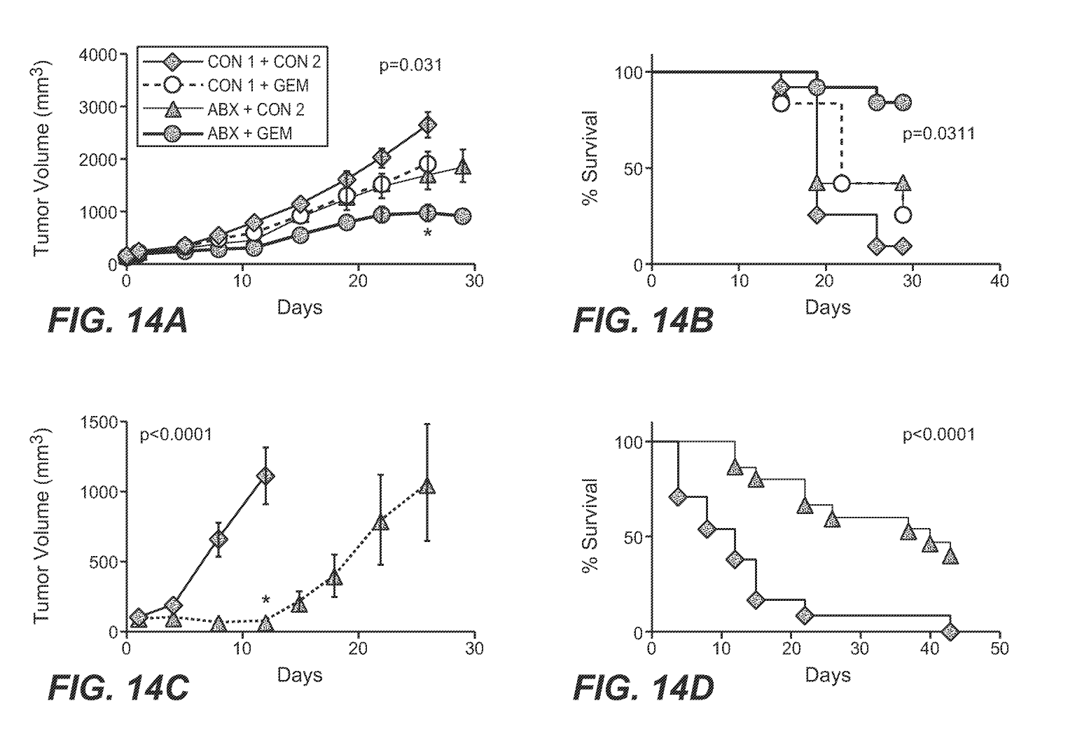

Growth inhibition was seen in all treatment groups, and the addition of Abraxane® to gemcitabine resulted in additive activity, (FIG. 14A, p=0.031 for combination compared to nab-paclitaxel alone; CON, control; GEM, Gemcitabine; ABX, nab-paclitaxel). The combination therapy prolonged survival (FIG. 14B; p=0.0311 for combination compared to nab-paclitaxel alone). Weight loss was <15% and the combination was tolerable.

Significant growth inhibition and improved overall survival was also seen in the Ewing sarcoma model with a single 5-day course of nab-paclitaxel alone vs. control (p<0.0001; FIGS. 14C and 14D; CON, control; GEM, Gemcitabine; ABX, nab-paclitaxel). Despite identical treatment regimens, growth inhibition from nab-paclitaxel was more pronounced in the Ewing sarcoma model than the osteosarcoma model, and so no additive benefit was seen with gemcitabine at the dosages used.

To perform statistical analysis, it was calculated that a sample of 10 mice per group would provide an 80% power to detect a difference in tumor size of 41%, Power was calculated at a significance level of 0.05 using a two-tailed, two sample Student's t-test assuming equal variance. GraphPad Prism 5 software was used to analyze survival by log-rank and tumor growth by two sample Student's t-test.

In summary, SPARC is expressed in the majority of Ewing sarcoma primary tumors, and particularly in recurrent tumors. When coupled with similar findings in osteosarcoma [8], this provides a biologic rationale for studying nab-paclitaxel in these tumors. Nab-paclitaxel also inhibited growth of osteosarcoma as reported earlier (Yang et al. “The efficacy of Abraxane on osteosarcoma xenografts in nude mice and expression of secreted protein, acidic and rich in cysteine.” American Journal of Medical Science 2012; 344:199-205), and gemcitabine appeared additive.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of treating a solid tumor in a human individual, comprising administering to the individual an effective amount of a composition comprising nanoparticles comprising a taxane and albumin, wherein the individual is no more than about 21 years old.

2. The method of claim 1, wherein the individual is no more than about 18 years old.

3. The method of claim 1, wherein the solid tumor is an abdominal tumor, a soft tissue tumor, a bone tumor, or an eye tumor.

4. The method of claim 3, wherein the solid tumor is a soft tissue sarcoma.

5. The method of claim 4, wherein the solid tumor is rhabdomyosarcoma.

6. The method of claim 1, wherein the solid tumor is neuroblastoma.

7. The method of claim 1, wherein the individual has had a prior treatment.

8. The method of claim 7, wherein the individual is resistant or refractory to the prior treatment.

9. The method of claim 7, wherein the individual has progressed on the prior treatment.

10. The method of claim 7, wherein the individual has a recurrent solid tumor.

11. The method of claim 7, wherein the prior treatment is a taxane-based therapy.

12. The method of claim 1, wherein the composition comprising nanoparticles comprising taxane and albumin is administered parenterally.

13. The method of claim 12, wherein the composition comprising nanoparticles comprising taxane and albumin is administered intravenously.

14. The method of claim 1, wherein the taxane is paclitaxel.

15. The method of claim 1, wherein the nanoparticles in the composition have an average diameter of no greater than about 200 nm.

16. The method of claim 1, wherein the taxane in the nanoparticles are coated with albumin.

17. The method of claim 1, wherein the weight ratio of albumin and taxane in the composition is 9:1 or less.

18. The method of claim 1, wherein the nanoparticle composition is administered at about 100 mg/m² to about 300 mg/m².

19. The method of claim 1, wherein the human individual is about 6 months to about 5 years old.

20. The method of claim 1, wherein the human individual is about 5 years old to about 9 years old.

21. The method of claim 1, wherein the human individual is about 10 to about 15 years old.
